(19) United States
(12) Patent Application Publication
(10) Pub. No.: US 2018/0001114 A1
(43) Pub. Date: Jan. 4, 2018

(54) AUTOMATED ULTRASOUND APPARATUS AND METHOD FOR NONINVASIVE VESSEL RECANALIZATION TREATMENT AND MONITORING

(71) Applicant: Vesselon, Inc., Norwalk, CT (US)

(72) Inventors: Rhodemann Li, Darien, CT (US); Clayton T. Larsen, Wilton, CT (US)

(21) Appl. No.: 15/536,482

(22) PCT Filed: Dec. 15, 2015
(86) PCT No.: PCT/US15/65837

§ 371 Date: Jun. 15, 2017

Related U.S. Application Data
(60) Provisional application No. 62/091,916, filed on Dec. 15, 2014.

(51) Int. Cl. A61N 7/00

(52) U.S. Cl. CPC A61N 7/00; A61N 2007/0078; A61N 2007/0004; A61N 2007/0039

(57) ABSTRACT

An apparatus and method for automatically lysing a clot to recanalize vessels using ultrasound, with or without nanoparticles, microbubbles, thrombolytic drugs or other agents, is described herein. At least one ultrasound transducer with a specified geometry, bandwidth and/or transmit pulse characteristics can generate a more uniform 3-dimensional volume of defocused acoustic energy into a target area of the body. When an agent (such as, but not limited to, microbubbles) is used to lyse a clot, a certain level of ultrasound energy may be desired in order to effect certain actions or states of the agent. Bone attenuation may present an issue inhibiting sufficient ultrasound energy from reaching the agent in order to attain the desired agent action. The relative patient dimensions and location of the clot may also present an issue.

### REFERENCE TO PENDING PRIOR PATENT APPLICATION

This patent application claims benefit of pending prior U.S. Provisional Patent Application Ser. No. 62/091,916, filed Dec. 15, 2014 by Vesselon, Inc. and Rhodemann Li et al. for AUTOMATED ULTRASOUND APPARATUS AND METHOD FOR NONINVASIVE VESSEL RECANALIZATION TREATMENT AND MONITORING (Attorney's Docket No. VESSELON-2 PROV), which patent application is hereby incorporated herein by reference.

### STATEMENT REGARDING FEDERALLY SPONSORED RESEARCH OR DEVELOPMENT

Not applicable.

### TECHNICAL FIELD

The present invention is generally directed to medical treatment methods, systems and apparatus, particularly those intended for treatment of disorders of the circulatory system, such as (but not limited to) brain circulatory disorders (e.g., ischemic strokes), cardiovascular circulatory disorders (e.g., myocardial infarction) and peripheral artery occlusions (e.g., arteriovenous fistula occlusions). More specifically, the present invention is directed to lysing a clot to recanalize a vessel using ultrasound, with or without nanoparticles, microbubbles, thrombolytic drugs and/or other agents.

### BACKGROUND OF THE INVENTION

In general, the sooner that a blood clot is dissolved or removed, the sooner that blood flow can be restored to the affected area of the body. Correspondingly, the faster that the blood flow is restored, the greater the likelihood of recovery of body function and/or survival. The present standard of care treatment for acute ischemic stroke patients in the hospital is the intravenous use of a thrombolytic drug, tissue plasminogen activator (tPA), for the dissolution of blood clots. For a variety of reasons, including time delays caused by the current need to rule out a hemorrhagic stroke, multiple contraindications, and increased risk of causing a symptomatic intracranial hemorrhage, tPA is administered to only approximately 5% of all ischemic stroke patients.

Sonothrombolysis is the use of ultrasound to augment the ability of a thrombolytic drug to break up a clot. Since tPA has a limited time window of effectiveness (i.e., approximately 3-4.5 hours) and the use of tPA can bring on severe adverse consequences (e.g., symptomatic intracranial hemorrhage, or death), researchers have sought to improve tPA's safety profile and effectiveness.

An improvement to sonothrombolysis, i.e., the use of microbubbles in conjunction with ultrasound to dissolve clots, has several origins. In 1995, Katsuro Tachibana, MD, PhD (First Department of Internal Medicine, Fukuoka University School of Medicine, Nanakuma, Japan) and Shunro Tachibana, MD, PhD (Sasguri Hospital, Japan) found through in vitro research that clot lysis (i.e., dissolution) with urokinase (a thrombolytic drug) was improved by using ultrasound. Urokinase's ability to dissolve clots was further enhanced by the combination of ultrasound and microbubbles. In 2000, it was also observed, by Andrei Alexandrov, MD (Director, Center for Noninvasive Brain Perfusion Studies, University of Texas Health Medical School, Houston, Tex.), that stroke patients undergoing intravenous (IV) tPA therapy with transcranial ultrasound Doppler monitoring had better outcomes than those patients with tPA and no ultrasound.

Microbubbles were known to increase the echo strength of ultrasound, especially in the brain which is protected by a thick skull bone that weakens the intensity of the ultrasound energy (i.e., by attenuation of the ultrasound energy). Researchers found that the combination of ultrasound with microbubbles and tPA improved the rate of clot dissolution (i.e., made clot dissolution faster and more complete than the use of thrombolytic drugs alone). Approximately 400 patients have been studied using ultrasound and microbubbles with tPA, and this combination has been found to be safe and more effective than tPA alone in peer-reviewed studies.

Because a common denominator of all of these human studies is tPA (with its concomitant adverse risks), others have sought to investigate, in animal models, what ultrasound and microbubbles can do without the presence of tPA. Animal studies have included pigs, rats, dogs and, in what is a preferred translational stroke model, rabbits. Eight peer-reviewed preclinical articles have demonstrated the utility, safety and effectiveness of microbubbles in conjunction with ultrasound in reducing infarct size, dissolving clots more completely and reducing the presence of intracerebral hemorrhage, either with or without thrombolytic drugs.

The physical mechanisms and processes by which microbubbles and ultrasound can work to dissolve a blood clot (thrombus) are multi-factorial, not fully understood and difficult to observe directly in vivo. One hypothesized interaction process is associated with the microbubbles achieving a stable cavitation state, i.e., where the microbubbles, under exposure to ultrasound energy, oscillate asymmetrically and do not burst. This oscillation is thought to generate mechanical energy around the microbubbles (referred to as microstreaming) which, researchers believe, promotes the motion of fluids to help to distribute tPA along the surface of the clot in order to dissolve the clot.

If the ultrasound energy delivered into the brain is too low to achieve stable cavitation, the ultrasound waves simply return a strong echo off the microbubbles, and may not provide the desired therapeutic effect. If, on the other hand, the ultrasound energy is higher than that which produces stable cavitation, the result may be that the microbubbles begin to implode (called inertial cavitation). This inertial cavitation, producing mechanical energy and microjetting of fluids, researchers theorize, may form pits on the surface of the clot, opening up more surface area for thrombolytics to work on dissolving the clot.

Researchers have also postulated a third mechanism where microbubbles may be able to disrupt the matrix-like fibrin lattice of a clot using what is referred to as radiation forces. It has been demonstrated in vitro that primary radiation forces associated with microbubbles, together with the effects of oscillation, may “push” microbubbles to form permanent tunnels to weaken and ultimately break up the fibrin lattice of a clot.

Stroke occurs around the world, afflicting people regardless of race, gender or age. Stroke patients are thereby heterogeneous, with treatment outcomes that may be affected by considerations such as (but not limited to) skull thickness, density or diameter; clot size, composition or age; and the relative level of the patient's health, cognition or function. Current ultrasound thrombolysis approaches, with or without microbubbles, are limited in their ability to automatically adjust or account for differences in patient-to-patient variations. For example, although the factors (such as attenuation) which affect how much ultrasound acoustic energy reaches an internal biologic region of interest, e.g., the middle cerebral artery (MCA), are known, there is a need to simply and automatically determine the level of attenuation occurring and to adjust the acoustic output energy so that the desired acoustic energy is delivered within the region of interest without the need to utilize an expensive and time-consuming diagnostic exam such as a head CT.

Vessel occlusive conditions may be life threatening if not resolved as quickly as possible. In the case of a cerebral vessel occlusion caused by a blood clot, the need to recanalize the vessel quickly becomes of paramount importance inasmuch as “time is brain.” First responders in the field, or emergency room personnel in the hospital, could save precious minutes (to hours) in treatment time, if an ultrasound thrombolysis system were noninvasive, automated and simple to use. Present ultrasound thrombolysis approaches, with or without microbubbles, are limited by their complexity, cost, or the need for time-delaying imaging prior to use (the imaging is required to identify the location of the occlusion).

First responders in the field, and emergency room personnel in the hospital, are often unable to treat stroke patients quickly enough to stave off fatal or debilitating effects. Since only a small percentage of ischemic stroke patients receive adequate acute treatment, there is need for faster, and more widely applied, immediate treatment systems and methods. A need exists for a noninvasive, automated, simple, and safe ultrasound thrombolysis system to treat or monitor the recanalization of an occluded vessel in an emergency setting or situation. To improve treatment outcomes, a need also exists for automatically collecting, recording or standardizing data from the many variables relating to a stroke patient's diagnosis, condition and/or treatment, beginning, ideally, at or near the onset of a vascular occlusive event and continuing through the conclusion of treatment.

The present invention overcomes the limitations of the prior art by also using at least one transducer, and at least one set of frequency, phase, timing and pulse parameters (provided by a signal generator/processor), in order to provide an area of uniform desired ultrasound energy levels. Furthermore the present invention automatically adjusts for, or accounts for, factors such as, but not limited to, backscatter, attenuation, beam aberration or the speed of sound. The present invention also automatically collects, records and/or standardizes the data associated with the many variables relating to a stroke patient's diagnosis, condition and/or treatment, beginning, ideally, at or near the onset of a vascular occlusive event and continuing through the conclusion of treatment, in order to improve treatment outcomes.

### SUMMARY OF THE INVENTION

One object of the present invention is to provide an immediate treatment, monitoring and/or diagnostic means for ischemic or occlusive conditions that addresses and overcomes the shortcomings and problems of the prior art.

In the situation where time is of the essence, such as for an ischemic event in the cerebral (e.g., stroke) or myocardial (e.g., myocardial infarction) vasculature, a noninvasive therapeutic ultrasound system, optionally including an agent, e.g., microbubbles, may be beneficially combined in kit form so as to enable minimally-trained personnel to more rapidly begin or deliver treatment to a patient. In situations where time-to-treatment may be less critical, a noninvasive therapeutic ultrasound system, optionally including an agent, e.g., microbubbles, may be beneficially combined in kit form to lyse a clot or dissolve an obstruction, such as, but not limited to, a deep vein thrombosis, arteriovenous fistula or graft obstruction. Additional applications of the present invention may include using an ultrasound system, optionally including an agent (e.g., microbubbles) as a means for drug or gene delivery. Depending on the location or condition being treated, different configurations of ultrasound transducer, operating parameters (provided by a signal generator/processor), and/or agents may be provided.

In one embodiment of the present invention, treatment is provided by at least three components: a transducer, a signal generator, and processor software (the signal generator and processor software may be incorporated in a single unit which is sometimes hereinafter referred to as a signal generator/processor). Additionally, the treatment may utilize an agent, such as microbubbles. The signal generator/processor settings may allow for pre-sets or patient-specific feedback of data in order to control the frequency, pulse length, duty cycle, acoustic output power, rate of energy alternation to transducers and/or other parameter combinations required in order to automatically deliver the optimal and appropriate acoustic energy through tissue, such as, but not limited to, the cranium and brain tissue. The acoustic energy provided is that which is appropriate to achieve the desired effect (e.g., the lysis of ischemic thrombus or embolus) with or without microbubbles.

Additionally, the present invention may include software to track, store and/or maintain records of any or all treatment episode digital log files associated with each patient and allow the uploading of additional patient-, purchaser- or user-related information. The digital log files act as a global archive of treatment episodes containing data of multiple parameters, logs and information, all of which can be secured for authorized user access control. Such data may be collected or analyzed by the processor (preferably provided as part of a combined signal generator/processor), or transmitted in real-time for off-site collection or analysis.

Accounting for ultrasound attenuation due to patient-to-patient variability with respect to tissue properties (e.g., skull thickness, diameter, porosity, density, etc.), or clot properties (e.g., composition, location, age, etc.) is provided herein.

Also, the present invention provides the ability to alter the depth of the energy intensity region provided by the ultrasound thrombolysis system.

Also, the ability to log data including, but not limited to, device settings, events, timings, and recordings, etc. of each treatment episode is also provided by the present invention.

In one preferred form of the present invention, there is provided an ultrasound system for providing therapy to a site in a patient, the ultrasound system comprising:

at least one transducer configured for removable securement to a patient; and

a signal generator/processor configured for removable connection to the at least one transducer, the signal generator/processor being configured to apply an electrical signal to the at least one transducer so as to cause the at least one transducer to deliver ultrasound energy to the site in the patient, whereby to provide therapy to the site in the patient;

wherein the signal generator/processor is configured to (i) identify ultrasound attenuation as the ultrasound energy passes through the patient, and (ii) modify the electrical signal applied to the at least one transducer so as to compensate for ultrasound attenuation in the patient, whereby to deliver a pre-determined level of ultrasound energy to the site in the patient.

In another preferred form of the present invention, there is provided an ultrasound system for providing therapy to a site in a patient, the ultrasound system comprising:

at least one transducer configured for removable securement to a patient; and

a signal generator/processor configured for removable connection to the at least one transducer, the signal generator/processor being configured to apply an electrical signal to the at least one transducer so as to cause the at least one transducer to deliver ultrasound energy to the site in the patient, whereby to provide therapy to the site in the patient;

wherein the at least one transducer and the signal generator/processor are configured to provide the ultrasound energy to the site in the patient in the form of a substantially uniform diverging beam.

In another preferred form of the present invention, there is provided a system for delivering ultrasound to a patient, the system comprising:

at least one transducer configured for removable securement to a patient; and

an adhesive for removably securing the at least one transducer to a patient;

wherein the at least one transducer and the adhesive are provided as a prepackaged kit.

In another preferred form of the present invention, there is provided a method for providing therapy to a site in a patient, the method comprising:

providing an ultrasound system for providing therapy to a site in a patient, the ultrasound system comprising:
at least one transducer configured for removable securement to a patient; and
a signal generator/processor configured for removable connection to the at least one transducer, the signal generator/processor being configured to apply an electrical signal to the at least one transducer so as to cause the at least one transducer to deliver ultrasound energy to the site in the patient, whereby to provide therapy to the site in the patient;
wherein the signal generator/processor is configured to (i) identify ultrasound attenuation as the ultrasound energy passes through the patient, and (ii) modify the electrical signal applied to the at least one transducer so as to compensate for ultrasound attenuation in the patient, whereby to deliver a pre-determined level of ultrasound energy to the site in the patient;

removably securing the at least one transducer to a patient, and removably connecting the signal generator/processor configured to the at least one transducer; and

applying an electrical signal to the at least one transducer with the signal generator/processor so as to cause the at least one transducer to deliver ultrasound energy to the site in the patient, whereby to provide therapy to the site in the patient.

In another preferred form of the present invention, there is provided a method for providing therapy to a site in a patient, the method comprising:

providing an ultrasound system for providing therapy to a site in a patient, the ultrasound system comprising:
at least one transducer configured for removable securement to a patient; and
a signal generator/processor configured for removable connection to the at least one transducer, the signal generator/processor being configured to apply an electrical signal to the at least one transducer so as to cause the at least one transducer to deliver ultrasound energy to the site in the patient, whereby to provide therapy to the site in the patient;
wherein the at least one transducer and the at least one signal generator/processor are configured to provide the ultrasound energy to the site in the patient in the form of a substantially uniform diverging beam;

removably securing the at least one transducer to a patient, and removably connecting the signal generator/processor configured to the at least one transducer; and

applying an electrical signal to the at least one transducer with the signal generator/processor so as to cause the at least one transducer to deliver ultrasound energy to the site in the patient, whereby to provide therapy to the site in the patient.

In another preferred form of the present invention, there is provided a method for providing therapy to a site in a patient, the method comprising:

providing an ultrasound system for providing therapy to a site in a patient, the ultrasound system comprising:
at least one transducer configured for removable securement to a patient; and
an adhesive for removably securing the at least one transducer to a patient;
wherein the at least one transducer and the adhesive are provided as a prepackaged kit;

opening the prepackaged kit;

applying the adhesive and the at least one transducer to a patient; and

using the at least one transducer to deliver ultrasound energy to the site in the patient, whereby to provide therapy to the site in the patient.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing a novel ultrasound thrombolysis system for applying ultrasound to the body of a patient so as to recanalize an occluded blood vessel;

FIGS. 1A-1C are schematic block diagrams showing operation of various ultrasound thrombolysis systems formed in accordance with the present invention, wherein the systems automatically adjust the energy level emitted by the system to compensate for ultrasound energy attenuation or aberration;

FIGS. 2A-2C are schematic views of various ultrasound transducers formed in accordance with the present invention which generate a defocused, 3-dimensional diverging beam pattern;

FIG. 3 is a schematic view of a low profile transducer with a radius of curvature generating a 3-dimensional cone of insonation covering a region of interest (versus the insonation pattern created by flat transducers);

FIG. 4A is a schematic view showing a contralateral approach for the detection of the attenuation of ultrasound energy through the skull or biological tissue;

FIG. 4B is a schematic view showing an ipsilateral approach for the detection of the attenuation of ultrasound energy through the skull or biological tissue;

FIG. 5A is a 3D graphic of a 10% bandwidth simulation, and an actual test plot with a narrow bandwidth continuous wave (CW) pulse, of a beam pattern of ultrasound energy generated by a 25 mm diameter transducer disk with a 100 mm convex radius of curvature, operating at 1 MHz; and

FIG. 5B is a 3D graphic of a 50% bandwidth simulation, and an actual test plot of a broad bandwidth single cycle pulse, of a beam pattern of ultrasound energy generated by a 25 mm diameter transducer disk with a 100 mm focal length, operating at 1 MHz.

### DETAILED DESCRIPTION OF THE PREFERRED EMBODIMENTS

In diagnosing, monitoring and/or treating time-sensitive conditions, the ability to (a) initiate treatment immediately (e.g., before imaging studies), (b) reduce manual steps or subjectivity, and/or (c) account for patient-to-patient variability, should lead to improved patient outcomes and processes. For an occlusive condition, such as ischemic stroke or myocardial infarction, the sooner a clot is dissolved and blood flow restored, the better the treatment outcome for the patient. The general concept of automation of a treatment to improve patient outcomes is generally known, however, and significantly, the present invention discloses various means to automate certain steps taken (i) during an emergency ischemic treatment process, and/or (ii) to account for differences in ischemic patient characteristics.

Regarding the automation of a treatment device, the present invention contemplates means to automate the delivery of the therapeutic ultrasound treatment as well as means to automate the level of ultrasound energy delivered within the region of interest, taking into account factors such as attenuation, backscattering and/or the speed of sound through different layers of biological tissue. Inasmuch as during the first response to an occlusive event, such as a stroke or myocardial infarction, a first responder will not know the exact location of the clot, the application of ultrasound energy should be evenly distributed to those anatomical regions that are at-risk candidates for harboring the blood-blocking clot. Also, since the ultrasound energy is attenuated by bone, the output energy of the ultrasound device may be different than the energy level that is actually delivered at the site of the clot, e.g., at some location in the Middle Cerebral Artery (MCA) or its immediate branches where a clot may reside. Skull bone thickness, density and porosity differ from person to person, thus affecting the amount of ultrasound attenuation and therefore the amount of energy actually delivered to the region of interest, e.g., the MCA. Having the desired amount of energy delivered to the site of the clot (e.g., into a length of the MCA) is important because certain ultrasound energy, with or without microbubbles, has been shown to promote clot lysis. Microbubbles have been shown, in the presence of certain ultrasound energy levels, to cavitate and/or radiate, thereby also promoting clot lysis. However, too much acoustic energy may be detrimental, while too little acoustic energy may not result in the desired therapeutic (or diagnostic) effect. In an emergency medicine situation where time (and cost) may be factors determining what treatments are applied, a simple, low-cost, easy to use and automated means to account for ultrasound attenuation, backscattering and/or the speed of sound is desired.

It is in the above context that preferred embodiments of the present invention are herein described.

### The Novel Ultrasound Thrombolysis System in General

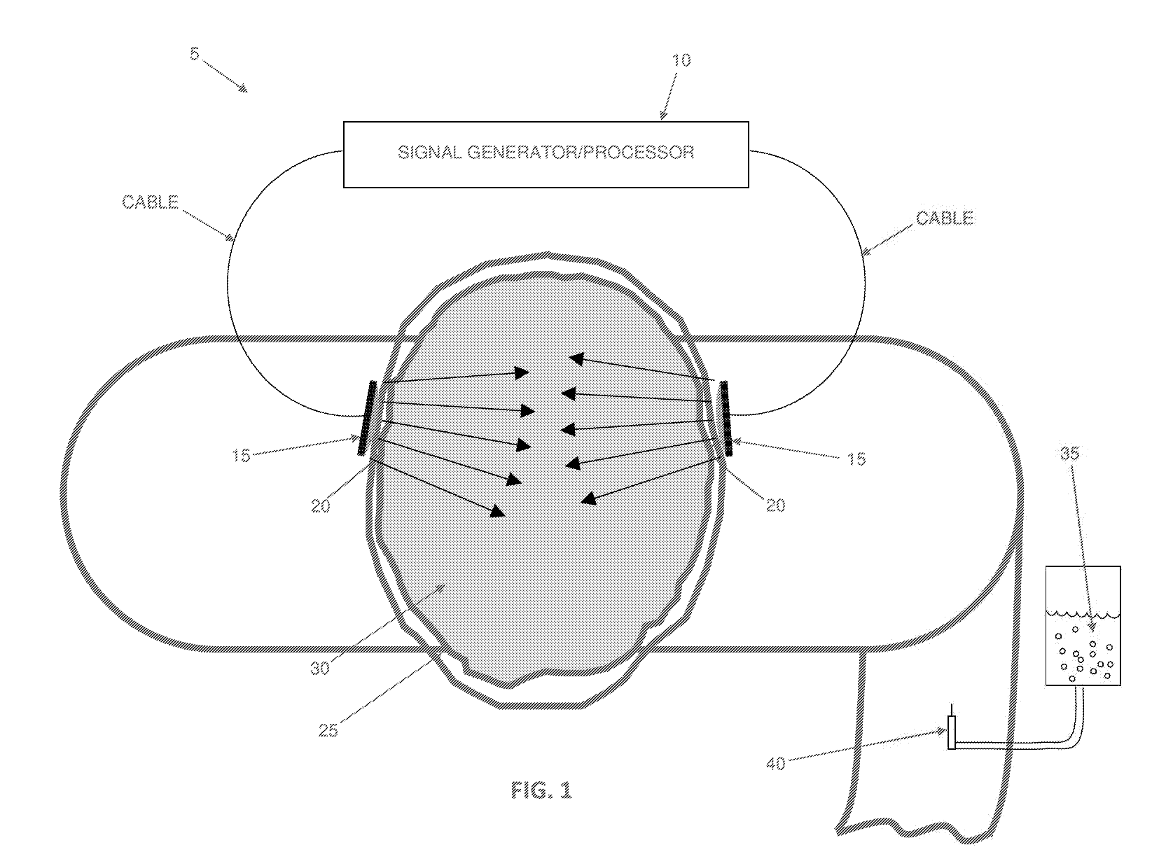

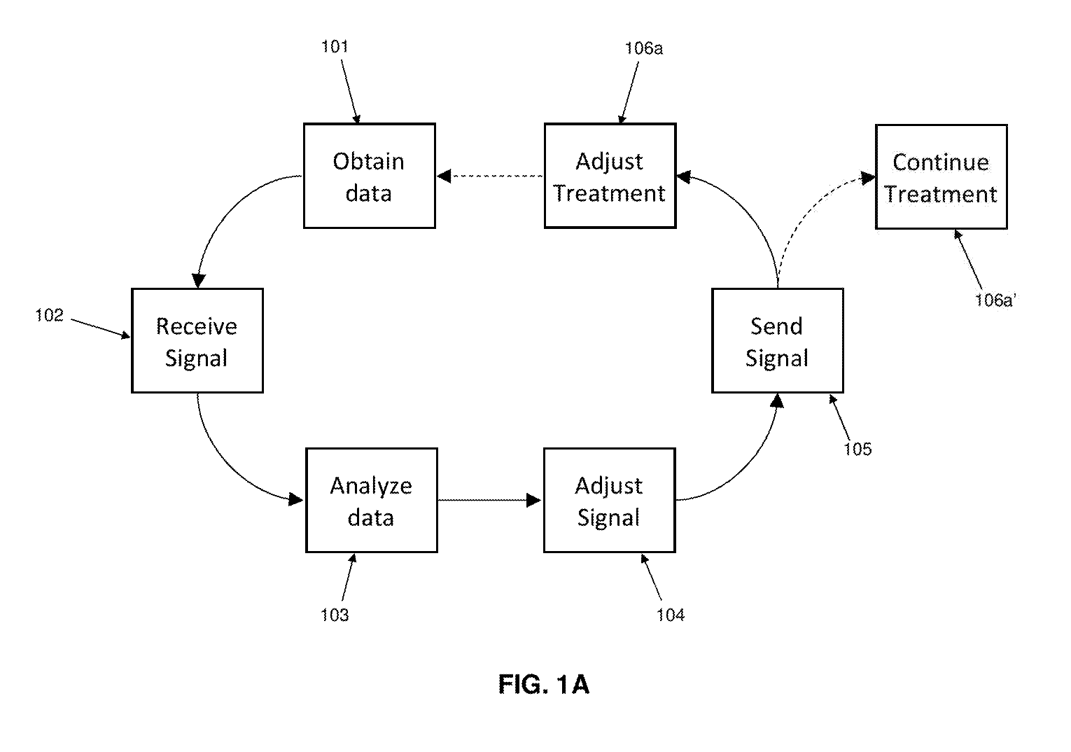

As will hereinafter be discussed, and looking now at FIG. 1, the present invention comprises the provision and use of a novel ultrasound thrombolysis system 5 for applying ultrasound to the body of a patient so as to recanalize an occluded blood vessel, e.g., a blood vessel in the brain during a stroke, a coronary artery during myocardial infarction, etc. Novel ultrasound thrombolysis system 5 generally comprises a signal generator/processor 10 for generating an appropriate electrical signal, one or more transducers 15 connected to signal generator/processor 10 for converting the electrical signal generated by signal generator/processor 10 into ultrasound waves, and an ultrasound-conducting medium (e.g., an appropriate gel) 20 for providing an appropriate interface between transducers 15 and the anatomy of a patient, e.g., the skull 25 of a patient overlying the brain 30 of a patient.

As will hereinafter be discussed, novel ultrasound thrombolysis system 5 may be used with or without an agent (e.g., microbubbles) 35, in which case a means 40 (e.g., a needle) to deliver the agent into a human or animal body may also be provided.

Details regarding the construction and use of novel ultrasound thrombolysis system 5, including the construction and use of its constituent components (i.e., signal generator/processor 10, transducers 15 and ultrasound-conducting medium 20), with or without an agent (e.g., microbubbles) 35 will hereinafter be discussed in further detail.

### Uniform Field of Coverage

A focused or unfocused ultrasound transducer emits a field of acoustic energy, typically in a planar fashion and with significant variations in acoustic intensity across the spatial width and depth of that field of view (FOV). Where the ultrasound energy is presented with a planar disposition, the user would need to know a priori where an occlusive clot was located, visualize it with imaging in real-time in order to correctly align the planar disposition of the ultrasound energy with the clot, and maintain the planar disposition of the ultrasound energy at that site (i.e., hold it in place) during the treatment duration (or repeatedly sweep the treatment across a targeted area). These requirements are extremely difficult to meet in a mission-critical emergency event where any elapse of time reduces the patient's likelihood of survival or disability-free quality of life.

A flat, single element unfocused ultrasound transducer will produce a diverging 3-dimensional cone with significant variations in acoustic intensity within the volume of insonation that are caused by peaks and nulls in the acoustic beam. In a preferred embodiment of the present invention, signal generator/processor 10 is configured to provide a swept and/or stepped frequency output signal of specific pulse duration to a 3-dimensional radiating defocused transducer 15 so as to provide a spatially diverse distribution of various peaks and nulls that, in sum, will contribute to forming a more uniform beam throughout the volume of insonation to interact with the clot and with the microbubbles (if they are present). In another embodiment of the present invention, by using a transducer 15 with a bandwidth of approximately 30% or greater, a succession of single half-cycle or full-cycle transmit pulses can be used to excite the natural response of the broad bandwidth transducer to generate a more uniform ultrasound field of energy to interact with the clot and with the microbubbles (if they are present in the vasculature within the region of interest).

### Correction for Bone Attenuation

A preferred embodiment of the present invention incorporates a feedback loop wherein the ultrasound thrombolysis system 5 and its use provides the ability to automatically adjust parameters such as, but not limited to, output energy, intensity, signal characteristics, pulse length, duty cycle, contrast agent size, distribution, composition, etc. to obtain a desired effect. A desired effect may be to control the output energy after accounting for ultrasound energy attenuation due to bone. Another desired effect may be to adjust for the depth of an area of uniform energy deposition after accounting for skull diameter. Yet another desired effect may be to adjust the output energy to account for the composition of an ischemic clot to be lysed.

FIG. 1A is a schematic block diagram showing operation of an ultrasound thrombolysis system formed in accordance with the present invention, wherein the system automatically adjusts the energy level emitted by the system to compensate for ultrasound energy attenuation or aberration. In this form of the invention, a step to obtain data 101 is initiated. Data such as skull bone characteristics (e.g, density, thickness, porosity, etc.) may be obtained by imaging or non-imaging means, for example, by sending an imaging or non-imaging interrogation signal (IS) into the skull bone and receiving back such signal data (e.g., by transmitting and receiving a signal at transducers 15), as shown at 102. Once signal data 102 has been received, a processor (e.g., signal generator/processor 10) analyzes, as shown at 103, the data 102, potentially adjusting the signal, as shown at 104, if necessary, and sending a new signal, as shown at 105. The new signal 105 may be an alteration of the treatment signal 106a, or may be a previously-established treatment signal 106a′. There may be a need to obtain data 101 again, in which case there may be a looped cycle.

FIG. 1B is another schematic block diagram showing operation of an ultrasound thrombolysis system formed in accordance with the present invention, wherein the system automatically adjusts the energy level emitted by the system to compensate for ultrasound energy attenuation or aberration. In this form of the invention, a step to obtain data 101 is initiated. Data such as skull bone characteristics (e.g, density, thickness, porosity, etc.) may be obtained by imaging or non-imaging means, for example, by sending an imaging or non-imaging interrogation signal (IS) and receiving back such signal (e.g., by transmitting and receiving a signal at transducers 15), as shown at 102. Once data 102 has been received, a processor (e.g., signal generator/processor 10) analyzes, as shown at 103, the data 102 and calculates an adjustment factor, as shown at 103b. Based on that adjustment factor 103b, a decision, shown at step 104b, will be made either to adjust the signal (as shown at 106b) if necessary or continue directly to treatment (as shown at 106b′).

FIG. 1C is another schematic block diagram showing operation of an ultrasound thrombolysis system formed in accordance with the present invention, wherein the system automatically adjusts the energy level emitted by the system to compensate for ultrasound energy attenuation or aberration. In this form of the invention, at least one ultrasound transducer 101c (i.e., the aforementioned ultrasound transducer 15) is used to transmit an interrogation signal through biological tissue, e.g., bone 110c. The interrogation signal may either traverse through the biological tissue 110c until at least a portion of the interrogation signal (IS) is received, at step 102c, by a second transducer 15 (not shown in FIG. 1C), or at least a portion of the interrogation signal (IS) may be reflected back and received by the transmitting transducer 101c. After at least one interrogation signal 102c is received by a transducer (either the transmitting transducer or a second transducer), a processor (e.g., signal generator/processor 10) analyzes the data, as shown at 103c, in order to calculate an adjustment factor, as shown at 104c. Based on that adjustment factor 104c, a decision (step 105c) will be made to either adjust the signal (step 106c) if necessary or continue directly to treatment 106c′ for diagnostic, monitoring and/or treatment purposes, which may include lysing an ischemic clot (not shown) with or without the use of an agent such as a microbubble (not shown).

Thus, as seen in FIGS. 1A, 1B and 1C, in one form of the present invention, ultrasound energy is delivered to tissue (e.g., the skull 25) via one or more transducers, e.g., transducers 15 of ultrasound thrombolysis system 5, and thereafter detected, either after traversing the tissue (in which case it may be detected by a second transducer) or after reflection back off the tissue (in which case it may be detected by the transmitting transducer). This detected ultrasound energy is analyzed (e.g., by signal generator/processor 10) and, based upon the analysis, an adjustment factor is calculated. This adjustment factor is used to determine whether the level of ultrasound energy should continue as is or whether the level of ultrasound energy should be modified.

### Treatment

Transducer

In one aspect of the present invention, a novel, small diameter, relatively flat (but somewhat curved) transducer (e.g., a transducer 15), operating around a center frequency (e.g., up to 10 MHz, or preferably around 1 MHz), may be used to keep the field of view (FOV) sufficiently wide to facilitate ultrasound thrombolysis. Examples of such a defocused, diverging ultrasound transducer include those made from a single convex curved crystal such as lead zirconium titanate, a composite material, or any material exhibiting a piezo-electric effect (“PZT”) that can transform electrical signals into mechanical energy and vice-versa.

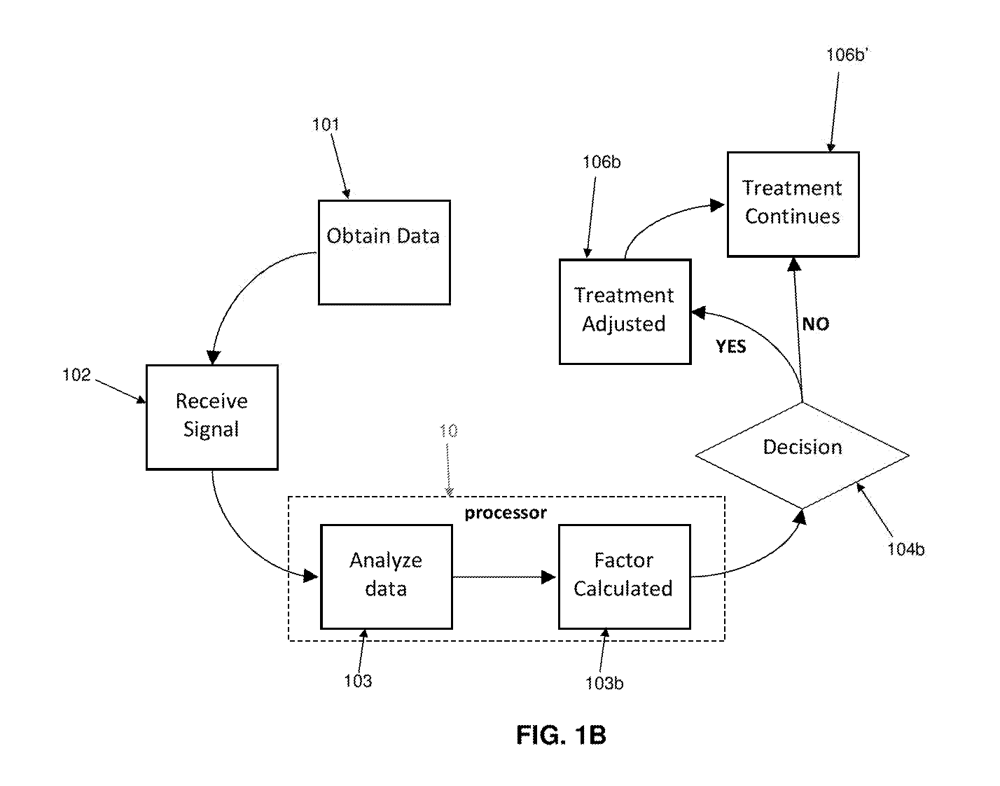

Looking next at FIG. 2A, there is shown an ultrasound transducer 15 formed in accordance with the present invention, using a lens material with a concave geometry 201. With this construction, an ultrasound signal emitting from the lateral edges of a round, oblong, square or other geometry transducer crystal 202 will traverse through a thicker lens layer 201, slowing down those sound waves. This creates a diverging spherical ultrasound wave (not shown) that spreads at depth away from the transducer. This is an example of a 3-dimensional diverging beam insonation geometry generated without phase beam formation or steering.

Looking next at FIG. 2B, there is shown another ultrasound transducer 15 formed in accordance with the present invention, wherein the ultrasound transducer 15 comprises a composite transducer 203 having PZT material diced into posts and then filled with epoxy. The composite transducer 203 may be electrically bonded as one element via gold sputtering, and the composite transducer 203 may then be formed under heat using a convex shaping tool 204 into a convex PZT geometry while maintaining single crystal operating characteristics. In essence, this forms a convex composite single element PZT transducer 205. This is another example of a 3-dimensional diverging beam insonation geometry generated without phase beam formation or steering.

Looking next at FIG. 2C, there is shown yet another ultrasound transducer 15 formed in accordance with the present invention, wherein the ultrasound transducer 15 combines an inverted concave acoustic lens 206 with the convex composite PZT transducer 205, with the two being joined such that the concave lens 206 conforms to the convex PZT 205. This produces a flat faced-appearing surface with incremental divergent beams greater than the lens or convexity can provide individually, and is yet another example of a 3-dimensional diverging beam insonation geometry generated without phase beam formation or steering.

Beam Pattern

A single diverging ultrasound transducer with a narrow bandwidth continuous wave (CW) pulse can generate side lobe effects that narrow the beam width at specified depths and introduce peaks and nulls within the desired field of insonation. In accordance with the present invention, by using a transducer (e.g., a transducer 15) with a bandwidth of approximately 30% or greater, a succession of single half-cycle or full cycle transmit pulses can be used to excite the natural response of a broad bandwidth transducer to generate a more uniform ultrasound field of energy, which is advantageous with ultrasound thrombolysis. This uniform field of energy may be used to interact with a blood clot (with or without the addition of microbubbles) in the vasculature within a region of interest. The depth range at which this uniform field of energy is generated may be adjusted based upon the frequency bandwidth and pulse characteristics used to excite the transducer.

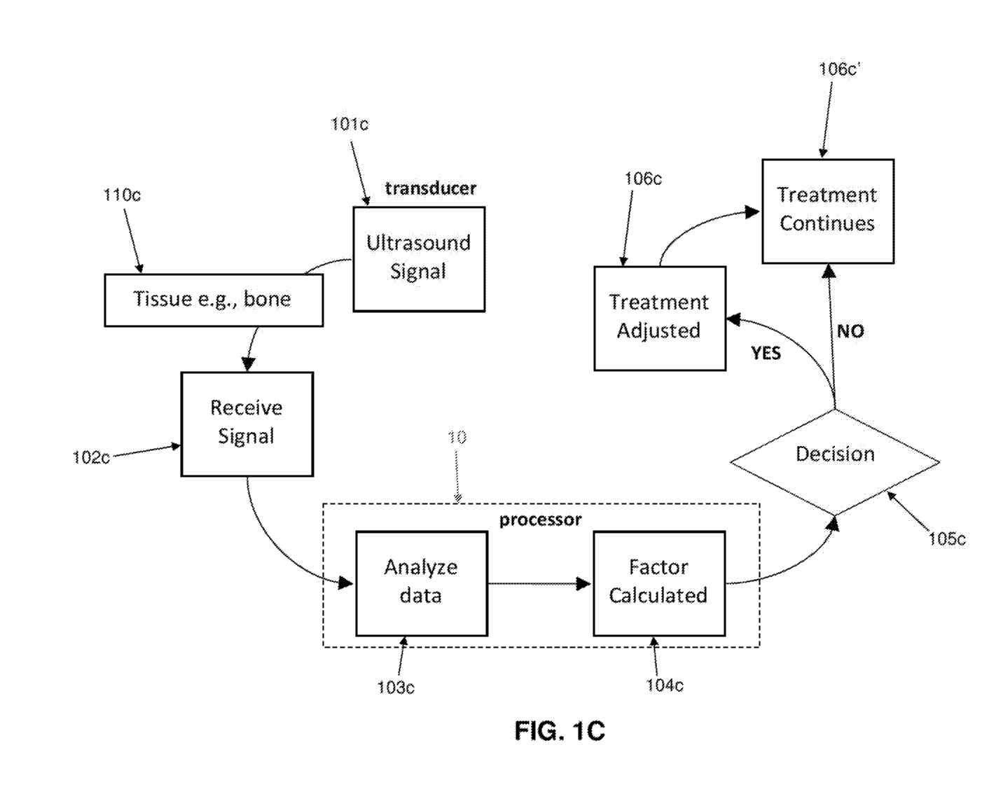

FIG. 3 shows a preferred embodiment of the present invention consisting of a single low profile transducer 301 (e.g., a transducer 15, preferably having a construction similar to that shown in FIGS. 2A, 2B and 2C) generating a cone of insonation 301a (ideally with a relatively uniform peak acoustic negative pressure at approximately 3 cm to 8 cm depth from the transducer) covering a region of interest 301b (represented by the shaded area in FIG. 3). This region of interest 301b may be where ischemic events are likely to occur and may include, in the case of stroke, the middle cerebral artery (MCA), penetrating arteries, and branches of the anterior cerebral artery (ACA) or posterior cerebral artery (not shown) at the so-called “Circle of Willis” in the brain.

The specific energy deposited into the region of interest 301b will be related to the output power of the transducer 301. However, due to attenuation or other factors, the actual energy that reaches the region of interest 301b may or may not be sufficient to lyse the blood clot (and/or to interact with injected agents such as microbubbles which may be circulating throughout the vasculature) so as to achieve desired effects on an ischemic clot, including lysis from radiation forces, stable cavitation and inertial cavitation. Any one or combination of these mechanical effects may also be used for other purposes, such as but not limited to, drug or gene delivery, or for diagnosis or monitoring purposes. As an example, inertial cavitation may be used to deliver targeted drugs to a site within a region of interest. Energy may be selectable or delivered automatically at different levels depending on factors such as, but not limited to the composition, age and size of the clot and, where microbubbles are being employed, the type, size and distribution of the microbubbles.

Researchers have used low frequency ultrasound for ultrasound thrombolysis in the cerebral vasculature because of significantly better penetration through the skull bone (compared to higher frequencies). Researchers have described using a 200 kHz flat unfocused disk of approximately 20 mm in diameter (as shown at 302) or 30 mm diameter (as shown at 303) that has a natural beam-spreading profile producing wide anatomical coverage for the targeted cerebral vasculature, the angles of which are described by the relation:

Θ=tan⁻¹(a/d)

a=transducer radius

d=far field transition where beam becomes conical

Therefore, a 20 mm diameter flat unfocused disk 302 may have a diverging beam pattern 302a that appears to more than cover the region of interest 301b, and a 30 mm diameter flat unfocused disk 303 may have a diverging beam pattern 303a that also appears to more than cover the region of interest 301b. However, a disadvantage of using very low frequency ultrasound (such as that used by researchers with the 20 mm disk 302 and the 30 mm disk 303) is the potential to open the blood brain barrier (BBB), which may cause serious adverse hemorrhagic events. Another disadvantage of using very low frequency ultrasound (such as that used by researchers with the 20 mm disk 302 and the 30 mm disk 303) is that the wide angle of divergence of the beam after the conical transition zone creates a rapid fall-off of acoustic intensity and a non-uniform deposition of acoustic energy within the region of interest. In some cases, there may be an almost five-fold reduction in energy over the distance of just 3 cm deep to 6 cm deep from the face of the transducer.

More Uniform Acoustic Energy in Region of Interest

Still looking now at FIG. 3, one preferred embodiment of the present invention uses a convex-formed PZT material (e.g., having a construction similar to that shown in FIGS. 2A, 2B and 2C) that will act as a single crystal transducer 301 (i.e., the transducer 15 of ultrasound thrombolysis system 5) that emits a conical or similar 3-dimensional volume of non-imaging ultrasound energy 301a to cover a region of interest 301b. The transducer 301 may have a diameter of approximately 25 mm or a similar cross-sectional dimension in an oblong, square or alternative geometry, and may have a radius of curvature to create a beam 301a of approximately 4 cm wide at 8 cm depth (covering, for example, the “Circle of Willis” at the midline of the brain of a stroke patient). Because the angle of divergence of the beam is more gradual with this form of transducer, the acoustic energy deposited in the vessel region of interest 301b may be more uniform compared to the acoustic energy generated by a sharply diverging beam, provided that other signal-generating techniques are used such as swept and/or stepped frequency output or broadband short pulses as described herein.

### Signal Generator/Processor

A preferred embodiment of the signal generator/processor 10 of ultrasound thrombolysis system 5 may be portable, lightweight, battery-powered, and may be provided in a form factor similar to that of a mobile computer, tablet or phone. The signal generator/processor 10 may have a digital display, touchscreen, keyboard, audio/visual components, microphone, antenna(e), or ports to receive or transmit analog or digital inputs/outputs. The signal generator/processor 10 may be detachably connected to the transducer(s) 15, or may transmit/receive data or signals to/from the transducer(s) 15 wirelessly. The signal generator/processor 10 may be connected to, integrated with or communicate with, a diagnostic, imaging, physiologic monitor/defibrillator or analytic device.

In one embodiment of the present invention, the signal generator/processor 10 is configured to log data such as, but not limited to, radio-frequency identification number (RFID #) generated data from each transducer 15, microbubble dose 35, or other component related to the treatment. As an example, the unique identification data may be recorded to track events, components, conditions, etc. after a patient is suspected of having a stroke and is transferred from a pre-hospital site to the emergency department of a hospital. This data may be recorded into the signal generator/processor 10 or into the transducers 15 and, furthermore, may be transmitted from the signal generator/processor 10 or transducers 15 to another location for archiving, evaluation, or processing. Certain components of the ultrasound thrombolysis system 5 (e.g., individually identified transducers 15, any dosage of agent 35 used) may be utilized in the field and may remain with the stroke patient even after the stroke patient is admitted into a hospital while maintaining their unique component identification, while other components of the present invention (e.g., signal generator/processor 10) with their own unique component identifier may be removably detached from the stroke patient, e.g., so as to allow emergency medical personnel to retain and re-use the signal generator/processor 10 for another stroke patient, even while the first patient is being treated or monitored in the hospital using a separate signal generator/processor (not shown).

For example, if treatment using the ultrasound thrombolysis system 5 is initiated in the field, then the transducers 15 would be adhered to the patient and contrast agent (if used) would be delivered directly or indirectly into the patient's circulatory system before arriving at a hospital. Once the stroke patient is admitted to a hospital those components of the ultrasound thrombolysis system 5 utilized in the pre-hospital setting may be detached from other components of the ultrasound thrombolysis system 5 to allow for the connection to those components of the ultrasound thrombolysis system 5 being utilized in a hospital setting. By way of example but not limitation, a transducer 15 affixed to the stroke patient out in the field may be left on the patient, but disconnected (if wired) from an ambulance-owned signal generator/processor 10, and the transducer 15 may be re-connected to another signal generator/processor 10 owned by the hospital in order to continue treatment for the patient and to allow the hospital-based signal generator to recognize the unique identification of the transducers that were applied to the patient when they arrived. The ability to “hand off” pre-identified components of the ultrasound thrombolysis system 5 (not limited to the transducers 15) with minimal treatment interruption is significant, particularly in situations where treatment is initiated out of the hospital and continued in the hospital, such as may be the case with stroke. Components having unique identifiers permit the transfer of event data relating to or the continuation of treatment through the use of the invention, particularly from one setting (e.g., prehospital) to another (e.g., hospital). Since the components have unique identifiers, an emergency medical service treating a stroke patient with its supply of the transducers 15, agent 35 and signal generator/processor 10 could begin treatment on stroke patient X, transfer patient X to the hospital, and disconnect its signal generator/processor 10 to prepare for possible treatment of a new stroke patient Y. In the meantime, the hospital could connect its own signal generator/processor (not shown) while accepting the transfer of any event data collected by the emergency medical service's signal generator/processor 10 or transducers 15 up to the moment of the signal generator swap out so that the hospital signal generator/processor can continue to monitor or treat patient X and capture ongoing event data.

In a further embodiment of the present invention, video/audio recording and interactive communication capabilities are provided in the signal generator/processor 10 for on-site neurological assessment or remote consultation, regardless of where the patient may be. The signal generator/processor 10 may have a video camera and audio microphone that can record and store visual and audio information, such as patient assessments based on, but not limited to, the National Institutes of Health Stroke Scale (NIHSS) or Cincinnati Prehospital Stroke Scale (CPSS). The display of the invention may be used to provide images from which an operator may query a stroke patient to gauge cognitive or motor function, as well as to record observations. In the present invention, an “in the field” ability to use a computerized means in order to administer an assessment test such as the NIHSS or CPSS may lead to more standardized procedures, improved scoring and enhanced documentation of treatment and quality of care.

In yet another embodiment of the present invention, the signal generator/processor 10 has the ability to receive data or signals from a remote location that in turn may allow for changes to its generator operating parameters. For example, since treatment using the present invention may be applied in a pre-hospital setting, data from the signal generator/processor 10 such as, but not limited to, NIHSS data entered by a user after treatment has been initiated may be interpreted by a physician (e.g., a neurologist via a telemedicine connection) and instructions may be transmitted to the signal generator/processor 10 in order to modify or cease treatment. Alternatively, embodiments of the present invention may utilize means to measure intracerebral blood flow, or proxies thereof, and data may be transmitted to a physician who may send instructions and/or data to the signal generator/processor 10 to modify or cease treatment.

Additional generated or inputted data, such as date or time, global positioning system (GPS), accelerometer, sensor, biological, chemical, demographic, patient-specific, functional or cognitive test information, or other data, may be incorporated into or otherwise utilized with the signal generator/processor 10 or transducer(s) 15.

Some illustrative examples of the signal generator/processor 10 and its functions include the provision and use of a date/time stamp to record the time that key tasks or steps are performed in the diagnosis, treatment or monitoring of a patient. For example, the signal generator/processor 10 may be used to record the time that ultrasound insonation, or microbubble delivery, began, as well as the duration of the same. The GPS feature may allow for the display or recording of the location of the stroke patient, distance to a desired or closest facility such as a certified stroke center or hospital, or estimated time of travel to such facility. The accelerometer may be used to assist in the measurement of, for example, the amount of arm drift in a physical assessment of stroke symptoms. The signal generator/processor 10 may also include sensors that can estimate, detect or measure a myriad of actions such as, but not limited to, intracranial pressure, hematoma, hemorrhage, ischemia, use or dosage of microbubbles, proper placement or alignment of the transducers, voluntary or reflexive horizontal eye movement (from the NIHSS standard), blood pressure, pulse rate, etc. Biological tests may be performed using aspects of the invention and incorporated into the diagnosis, treatment or monitoring functions of the invention, such as for the detection or measurement of blood glucose concentration, platelet count, current use of anticoagulant or direct thrombin inhibitors. The signal generator/processor 10 may have input/output ports to allow for the use or collection of the abovementioned data or tests.

### Registry Clinical Software

The collection of data during the use of the present invention can provide valuable insight into the progression or treatment of a biological condition, such as stroke. For example, the ability to track time and duration of treatment and to correlate such information to reduction in NIHSS scores during treatment may, in conjunction with patient-specific data such as skull morphology, lead to improvements in the timing and nature of treatment algorithms and processes. The data may be archived, analyzed, processed or transmitted on a HIPAA authorized and encrypted basis to pre-identified users with associated access credentials. Event data collected by the signal generator/processor 10 or the transducers 15 during each use of the invention creates an ever-increasing database that may be analyzed for trends, to develop best practices based on different treatment variables, comparison of local results to global results, etc. It is believed that payors or providers would value insights gained from the analysis of the data.

### Preparing for Treatment

The transducer(s) 15 may be packaged separately, or together with, other components of the ultrasound thrombolysis system 5, such as with a coupling gel or adhesive paste (e.g., the aforementioned ultrasound conducting medium 20), and/or microbubble dosage (e.g., the aforementioned microbubbles 35), and/or signal generator/processor 10. If packaged in a kit, the transducer/adhesive combination may be contained in a peel-away pack allowing for transducer placement onto a stroke patient's scalp to deliver acoustic energy through the hair and scalp and into the temporal bone window. Additionally, the microbubble single dose packaging may carry an RFID that records the dosage amount, manufacturer and lot number in a fixed or peel away format so that it can be adhered to an intravenous fluid (IV) bag and communicate with the ambulance signal generator/processor 10, or the switched-over-to the hospital-owned signal generator/processor 10 once the patient has reached the emergency department.

A kit may be provided, for convenience, containing (a) at least one ultrasound transducer 15, (b) a means to removably attach the transducer to the patient (e.g., ultrasound conducting medium 20), (c) an agent (e.g., such as, but not limited to, a nanoparticle, microbubble, thrombolytic drug or other agent solution, such as microbubbles 35), and (d) a means to deliver the agent into a human or animal body (e.g., a needle 40) so as to deliver a therapeutic effect or to deliver drugs. The ultrasound transducer(s) 15 may be of varying configurations, suitable for a particular treatment or diagnostic indication, e.g., a transducer may be provided as a single transducer element or as an array of transducer elements. The means to removably attach the transducer to a patient may itself be attached to the transducer, or the means to removably attach the transducer to a patient may be separate from the transducer and may be applied either first to the patient and then the transducer or to the transducer first then to the patient, e.g., as a paste, adhesive, glue, gel, or in a bandage form. The agent may be in an existing form ready for delivery into a patient, or may be a microbubble solution, other solution, suspension, powder or other form that may be constituted into an agent at the time of delivery into a patient. The means to deliver the agent into a patient may be a syringe, tube, intravenous port, hydrogel, gel, transdermal patch, aerosol, or other means. Furthermore, the delivery of the agent may include a means to agitate the solution containing the agent to ensure that the agent will be delivered into the patient and not remain in the solution outside of the patient, e.g., a mechanism (or audible/visual signal to alert a user) to agitate the intravenous saline bag containing the agent so as to ensure that any agent material floating above the delivery port due to a lighter weight relative to the saline solution can be delivered into the patient.

The present invention contemplates a rapid means for an emergency first responder to adhere one or more transducers 15 to the patient's scalp in a position that, for some patients, lies in hair or on the hairline. For ultrasound to be effectively transmitted into the body, a coupling gel (e.g., ultrasound conducting medium 20) having no air bubbles is typically used. This is because air would reflect the ultrasound waves, compromising the ability of the ultrasound energy to reach the region of interest. The coupling gel is ideally “acoustically transparent” in that there is little-to-no attenuation of the ultrasound energy due to the coupling medium so that the entirety of the ultrasound wave can be transmitted into the body as efficiently as possible. Most ultrasound coupling materials are viscous gels that are also quite slippery, enabling a traditional hand-held ultrasound transducer to glide over the body during a diagnostic exam.

In the present invention, there is provided not only a means to optimally transmit ultrasound through the skull, but there is also provided a means to couple a single element transducer to the patient's scalp through the hair (without the need to remove hair before applying the transducer). The transducer typically needs to be fixed in place for a period of time, which may be up to an hour or more for treatment.

To this end, the present invention contemplates the use of an adhesive paste or hydrogel (i.e., ultrasound conducting medium 20) that also possesses properties that allow for the ultrasound energy to penetrate through the hair and into the scalp. The adhesive may be a mixture formulated to take out insoluble particulates, while maintaining adhesion and ultrasound transmission properties through the hair to the skin and obviating the need to shave hair from the patient in order to initiate the application of ultrasound energy to the patient. Formulation of the adhesive also preferably includes a degassing process so that there is no trapped air within the adhesive. Properties and relative quantities of components may be optimized to achieve a low attenuation of ultrasound energy, including but not limited to ultrasound energy approximately around 1.0 MHz frequency.

Ingredients for the adhesive (i.e., ultrasound conducting medium 20) may include: hydrogel, olyoxyethylene 20 cetyl ether, water, glycerin, calcium carbonate, 1,2 propanediol, polyoxyethylene 20 sorbitol, methylparaben, or propylparaben. The invention contemplates using an amount of acoustic adhesive that will cover the entire face of the transducer(s) 15, even if the transducer has a curved face to accommodate a diverging beam configuration (see FIGS. 2A, 2B and 2C). The adhesive at the surface of the scalp will also serve as a flexible acoustic standoff, allowing for adjustment of the transducer(s) 15 in both lateral and angular alignment so as to allow the transducer(s) to be aligned with the anatomical region of interest (as well as to facilitate alignment of ipsilateral and contralateral transducers on opposing sides of the skull, see below).

### Activation

Unlike approaches that may rely on imaging data (time-consuming) or interpreting harmonic signals to detect microbubble cavitation states (complex or difficult to assess), the present invention uses means to calculate the actual amount of energy degradation for a particular patient in situ and automatically adjusts the acoustic output energy at a transducer to achieve a desired acoustic energy within a region of interest.

### Power Customization for Patient-Specific Skull/Bone Attenuation

Prior art approaches for estimating or calculating the effects of the skull on ultrasound beams are primarily concerned with how a focused ultrasound beam is aberrated by the skull. Various means are then used to account for this aberration and to correct for it in order to preserve beam-focusing on targets inside the brain. Preferred embodiments of the present invention involve non-imaging, wide field of view (FOV) treatment for stroke, obviating the need to utilize beamforming to any degree. One aim of the present invention is to introduce ultrasound energy into a wide region of the central brain vasculature to interact with injected microbubbles. Image formation or focusing is not required. The present invention contemplates means to manually or automatically regulate the amount of acoustic energy deposited within brain tissue (as compared to energy output from the transducer), such that the peak intensities of the acoustic energy are kept at or below maximum levels identified by the American Institute of Ultrasound in Medicine (AIUM) or United States Food and Drug Administration (FDA) which are considered to be safe to prevent harmful bioeffects. The invention also aims to keep acoustic intensities within a range that best interacts with blood clots and/or with microbubbles to enhance their therapeutic effect on blood clots. In a swept and/or stepped frequency embodiment, a variable voltage “map” of the transmitted pulses may also be employed for uniform acoustic energy deposition. More particularly, for any specific single element transducer design, no matter how broad the bandwidth of its PZT, the transducer will have differing efficiency characteristics when excited at various frequencies. This efficiency variation by frequency can be accounted for by exciting each frequency pulse at a different voltage such that the resulting beam generates an equivalent acoustic intensity in a free field. This standard output variable voltage map may then corrected for, based upon individual interrogation signals at each frequency.

Thickness, Density, Porosity, Diameter Via Interrogation Pulse

The present invention provides means to automatically set acoustic intensity levels that lyse a blood clot and, where microbubbles are used, may induce stable cavitation, inertial cavitation and radiating penetration in combination. These means are based on known attenuation coefficients of certain types of tissue along with the idiosyncratic nature of the anatomy of the skull compared to other parts of the body. In typical diagnostic ultrasound where an image is formed, the differential attenuation of the ultrasound beam as it transmits through various levels of tissue allows a line of information to be constructed in real-time. It is known that skin, fat, muscle, organs and bone attenuate to a different degree and can therefore be discretely reconstructed in an image plane. The temporal window of the skull provides both a simpler and yet more challenging environment for ultrasound. It is a simpler model because of the lack of fat and muscle in this region and, since skin has such a minute attenuation compared to the adjacent skull bone, it can be eliminated in any modeling of beam characteristics. What is left are the two main components, skull bone and brain tissue. Brain tissue has been found to be uniform in ultrasound attenuation and generally consistent from human to human, with such attenuation being −0.8 dB/cm/MHz. The remaining attenuation is due to skull bone, which varies significantly from patient to patient in terms of how much attenuation of an ultrasound beam it introduces. In addition, there are three main characteristics of the skull which each contribute to its attenuation profile: skull thickness, skull density and skull porosity.

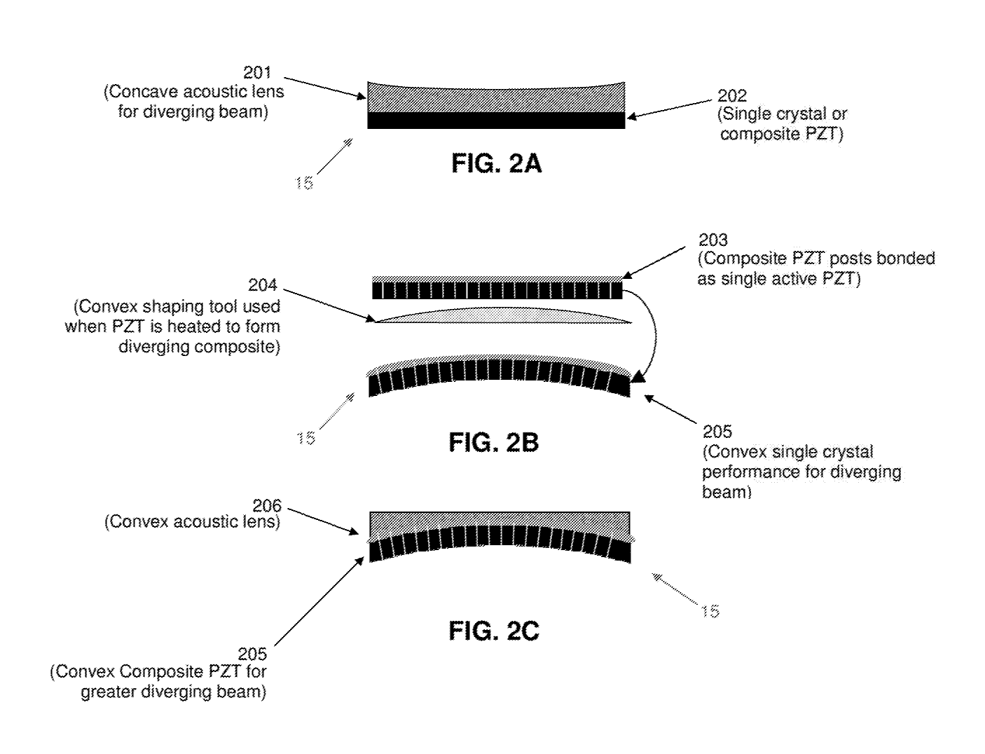

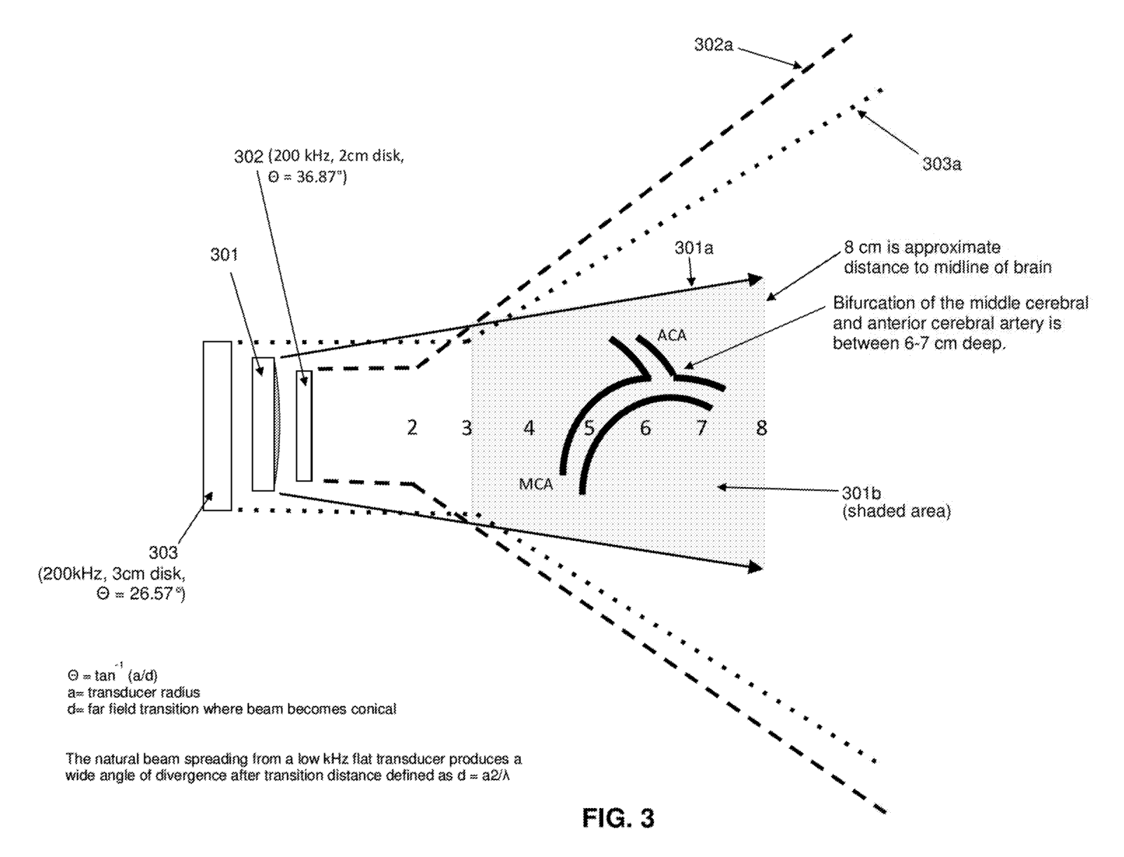

A set of calibration blocks formed out of attenuating material which mimics varying skull thickness, or cadaveric skulls can be set up in a test fixture of degassed water with hydrophone sensors to measure the peak negative pressure exhibited at several areas within the cranium compared to the transducer output at the ipsilateral, external part of the temporal bone and the acoustic signal received by a transducer on the contralateral, exterior temporal bone (see FIG. 4A, discussed in greater detail below). By taking measurements of acoustic intensity at multiple spatial points representing, for example, the target volume of insonation as well as acoustic intensity at the contralateral transducer, a tabular set of data is constructed for multiple locations on skulls of differing attenuation characteristics. A best-fit analysis of that data creates a look-up table and/or algorithm that accurately estimates the energy intensity at the interior cranium target area based solely upon the level of acoustic signal received at the contralateral side of the skull. This in vitro-derived algorithm is then compared to data from normal human volunteers (who have had a recent CT head scan) where contralateral transmission data is acquired without the benefit of inserting a hydrophone into the subject's brain. This transmission data is then generally correlated to known CT data that measures skull thickness and density to confirm the correlation to in vitro data.

### Contralateral Transmission Attenuation Detection

In accordance with the present invention, when Interrogation Signals (IS) with certain characteristics (including but not limited to multiple swept and/or stepped frequencies that match the therapeutic swept and/or stepped frequencies) are sent from an ipsilateral transducer 15 and (i) received at a contralateral transducer 15 (see FIG. 4A, discussed in greater detail below), or (ii) received back at the same ipsilateral transducer 15 (see FIG. 4B, discussed in greater detail below), the received IS will give specific data associated with that particular patient's skull such as, but not limited to, attenuation due to frequency, thickness, density and porosity. Each skull property contributes to bulk ultrasound attenuation which represents the major effect on a defocused ultrasound beam which cannot be calculated based upon a visual or quantitative analysis of a CT scan. The measured bulk attenuation can give real-time feedback which can be used to increase or decrease the output power control of the defocused transducer(s) and can thus be determined in the field and prior to any in-hospital diagnostic tests. These bulk attenuation measures are specific to that patient's skull, specific to the transducer placement at that point of attachment to the scalp and specific to the selected frequencies employed in a swept and/or stepped frequency pulse schema.

Looking next at FIG. 4A, there is shown an embodiment of the present invention relating to automatic attenuation detection using contralateral detection of an Interrogation Signal (IS). Electrode-like transducers 401a, 401b (e.g., transducers 15 of ultrasound thrombolysis system 5) are placed on a body, e.g., for stroke treatment on each opposing temple which typically affords the thinnest area of the skull for the optimal acoustic window into the brain. A signal or short set of IS pulses 401a′, 401b′, which may be delivered by a transducer 401a, 401b or other device, different from the subsequent treatment insonation pulses (but well within AIUM/FDA safety limits) is transmitted from one transducer 401a and received at the contralateral transducer 401b and measured in terms of its attenuation (or, alternatively, is transmitted from one transducer 401b and received at the contralateral transducer 401a and measured in terms of its attenuation). This attenuation is compared to a reference baseline derived from in vitro and in vivo test data described above. A compensation coefficient (also sometimes referred to herein as an “adjustment factor”, such as adjustment factor 103b, adjustment factor 104c, etc.) from the reference is derived or calculated. Attenuation from skin and thin muscle/fat layers at the scalp is negligible compared to skull bone and multiple centimeters of brain tissue and may be ignored. For calculation purposes, it is assumed that brain tissue attenuation α=−0.8 dB/cm/MHz is constant in normal brain tissue from patient to patient. Thus, the coefficient correlates well to the attenuation characteristics of the patient's specific skull compared to the baseline, taking into account the in situ transducer placement and exact skull location. The compensation coefficient is fed back to the signal generator/processor 10 so as to modify operation of the signal generator/processor (see for example FIGS. 1A, 1B and 1C). More particularly, if the compensation coefficient is greater than 1.0, the signal generator/processor 10 will increase the acoustic output given the higher detected attenuation versus the baseline measurement. However, if the compensation coefficient is less than 1.0, the signal generator/processor 10 will decrease the acoustic output given the lower detected attenuation versus the baseline measurement. In this way, the ultrasound energy output of tranducer(s) 15 may be tailored according to the patient-specific ultrasound attenuation so as to provide a desired level of ultrasound energy at an interior target site.

In one embodiment of the present invention, an IS pulse 401a′ from the first transducer 401a is transmitted to the second transducer 401b, and the compensation coefficient is calculated.

In another embodiment of the present invention, an IS pulse 401b′ from the second transducer 401b is transmitted to the first transducer 401a, and the compensation coefficient is calculated.

In still another embodiment of the present invention, an IS pulse 401a′ from the first transducer 401a is transmitted to the second transducer 401b, and a first compensation coefficient is calculated, an IS pulse 401b′ from the second transducer 401b is transmitted to the first transducer 401a, and a second compensation coefficient is calculated, and then an averaged compensation coefficient is calculated (i.e., by averaging the first compensation coefficient with the second compensation coefficient), and the system utilizes the averaged compensation coefficient to tailor the ultrasound energy delivered to an internal target site.

Additional IS may be transmitted at any time during the treatment insonation period as a check to see if anything has changed compared to the initial IS. Changes to the compensation coefficient may be caused by events such as, but not limited to, transducer movement or misalignment, changes to a clot, blood flow or pressure characteristics.

Any attenuation correction (AC) algorithm (i.e., to calculate a compensation coefficient or adjustment factor) may only be able to handle a transducer misalignment range that creates an approximate −1 dB offset from peak alignment. This misalignment range has been tested and found to be approximately 3 degrees angular and 1.3 cm lateral displacement with current transducer geometry. If, for example, transducer misalignment resulted in a −3 dB signal from actual peak (about 50% below peak amplitude), then the AC algorithm could provide a compensation coefficient or adjustment factor which could cause the signal generator/processor 10 to try to increase the power by 100% to compensate. A solution to potential transducer misalignment is as follows: the user manually aligns the transducers 15 on either side of the scalp with some audible or visual feedback (as described below using an IS). While the user is in the process of aligning the transducers 15, the signal generator/processor 10 continuously transmits an IS and records the received ultrasound amplitude signal and provides a visual or audible signal relating to the peak intensity. When the user reaches a peak alignment (which the system automatically records and measures without user input), the user can continue to press on the transducers 15 with the adhesive (i.e., ultrasound conducting medium 20) to get the optimal adhesion and placement to the scalp, even if the user moves the transducers by an angle from the peak received amplitude point. Even if the alignment of the ipsilateral transducer with the contralateral transducer goes off from the peak, the 3 cm-6 cm region of interest corresponding to the Middle Cerebral Artery will still be well within the angle of the transducer insonation 3D volume on the ipsilateral side for the therapeutic pulses. The system can then take the attenuation measurement from the calculated peak alignment point that was previously recorded and use that attenuation measurement as the correction factor (also sometimes referred to herein as the “compensation coefficient” or the “adjustment factor”) because that would best represent the actual skull attenuation encountered at that specific adhesion point on the scalp.

### Ipsilateral Attenuation Detection Via Backscatter

In order to avoid any potential inconsistencies of attenuation assumptions across the brain, or if some trauma or surface irregularity prohibits adhesion of the contralateral transducer, the following power customization process uses only a transducer (e.g., transducer 15 of ultrasound thrombolysis system 5) located on the ipsilateral side of the brain. More particularly, and looking now at FIG. 4B, there is shown an alternative embodiment of the present invention wherein a transducer 15 (i.e., transducer 402a, 402b) is placed on one or both temples. At the beginning of treatment, a pulse echo test, an IS, can be performed on a single transducer (i.e., either transducer 402a or 402b) to measure the relative attenuation of the intervening skull on that side of the brain. To do this, an IS 402a′ or an IS 402b′ is emitted via transducer 402a or 402b, respectively, the received signal is time-gated and its magnitude measured by that same transducer which emitted the IS. It has been demonstrated that normal brain tissue has a particular constant backscatter coefficient η=1/cm, just as it has a characteristic attenuation coefficient. By setting the time gate at a limited window, only echoes received back from the proximal part of the brain tissue will be received and these are unlikely to have been affected by any stroke disease processes occurring in the vascular tree closer to the midline. The magnitude of backscatter-returning echoes can then be compared to a baseline that can be generated from normal volunteers that have CT scan data available as described above in the contralateral attenuation correction embodiment (see FIG. 4A). A power modulation coefficient (also sometimes referred to herein as a “correction factor”, a “compensation coefficient” or an “adjustment factor”) can be derived that either increases or decreases the power delivered to the transducer on that side of the brain during the remaining time of the treatment. This process may be repeated on the opposite transducer and a separate power modulation coefficient can be developed for that hemisphere of the brain if the skull surface allows.

The benefits of this approach include, but are not limited to: no alignment is required between opposing transducers on each temple; no intervening interior brain pathology affects this relative quantitative approach; and any asymmetry between the shape, thickness, density or porosity of each temporal bone is accounted for individually so the power is customized not only by patient but also by hemisphere of the brain.

Any contralateral approach using highly focused or fixed focus transducers to detect an IS sent by an ipsilateral transducer is likely to be highly dependent on alignment of the two transducers, not just during the attenuation correction calculation but also during the entire treatment duration, due to the limited 3-dimensional coverage and non-uniform acoustic energy distribution. Regardless of the type or number of transducers used, as long as the IS can be received and interpreted, whether using a contralateral IS-transmission approach (see FIG. 4A) or an ipsilateral IS-reflection approach (see FIG. 4B), aspects of the present invention may be used to correct for ultrasound attenuation and/or aberration.

The contralateral or ipsilateral approach may be limited by the assumption of uniform attenuation across the brain of a specific patient as well as by the assumption that inter-patient variability of brain tissue attenuation is insignificant. The latter assumption appears to be supported in that normal brain tissue has a relatively uniform attenuation across different patient populations. The former assumption, however, may introduce some very small estimation inaccuracy in patients with cerebral pathology, either associated with the stroke that is being treated or by a pre-existing cerebral abnormality such as a mass or edema. If any significant brain cell death or edema has occurred within the ultrasound path between the opposite transducer electrodes in the contralateral approach, there may be some modest but measurable variability in the patient attenuation data compared to the baseline attenuation data. This percentage variability in intracranial soft tissue masses/fluids is likely to be insignificant as compared to the overwhelming magnitude of attenuation represented by the skull bone, which can reach over 90% signal loss at frequencies in the diagnostic range of ultrasound. Edema, or pooled blood, or a shifted midline due to a mass, all have very similar attenuation coefficients to soft tissue (e.g., brain). So the presence of internal cerebral pathology should not be any significant source of variability.

### Adjustments for Patient Skull Width Variations

An interrogation signal (IS) with certain characteristics sent from a first transducer 15 and received at a second contralateral transducer 15 will give specific “time-of-flight” (TOF) data associated with that patient's skull such as, but not limited to, the diameter of the head between opposing transducers. If this measurement shows that the patient has an unusually narrow or wide head, outside predefined anthropomorphic norms, it can be assumed that the cerebral vascular targets for treatment will be more shallow or at a greater depth than normal subjects. This head size data may optionally be used to additionally vary the input signal to the transducer. The depth range at which a uniform field is generated can then be adjusted to be closer to the transducer if the measured head size is of a particularly narrow size or can be adjusted to begin at a greater depth from the transducer if the measured head size is particularly large. This patient-specific depth adjustment will then provide the uniform partial cone of insonation that would best align to where their target cerebral vessels lie within that particular skull of that patient. For example, the present invention may use a specific pulse length, duty cycle and bandwidth frequency content to provide a uniform acoustic energy distribution in a cone from the transducer face to the brain midline at approximately 8 cm deep. The particular baseline target range for the M1, M2 and Lenticulostriate arteries may be, for example, a conical section from 3 cm to 6.5 cm deep that the beam uniformity is optimized for. If, however, the time-of-flight (TOF) data indicates that the patient has a narrower or wider skull/brain, the feedback sensor from the time-of-flight (TOF) calculation can alter the ultrasound parameters (e.g., specific pulse length, duty cycle and bandwidth frequency) so that the optimally uniform conical section is, for example, 2.0-5.5 cm or 4.0-7.5 cm.

For the power adjustment feedback described above (i.e., calculation of the “power modulation coefficient”, “correction factor”, “compensation coefficient” or “adjustment factor” to account for ultrasound attenuation), the amplitude of the IS is used for setting the patient-specific acoustic output level. In addition to amplitude, a time-of-flight (TOF) measurement can also be made from the same IS to provide an accurate measure overall width of the patient's head. If the head (and thus skull) are found to be narrower or wider than a predetermined threshold, the TOF width factor can be used as a feedback signal to the signal generator/processor 10 so that the signal generator/processor modulate the signal sent to a transducer (i.e., modulate the specific pulse length, duty cycle and bandwidth frequency) so as to adjust the disposition of the uniform ultrasound field emitted by the transducer.

In other words, the signal generator/processor 10 can vary the depth of the uniform acoustic field by changing the signal characteristics transmitted to the transducer (i.e., by modulating the specific pulse length, duty cycle and bandwidth frequency). If the TOF calculator determines a narrower head, the signal generator/processor 10 will adjust the output signal sent to the transducer to move the uniform acoustic field closer to the transducer so that it may more accurately match the patient's shorter hemisphere width and closer cerebral vasculature. If the TOF calculator shows a wider head, the signal generator/processor 10 will adjust the signal sent to the transducer and hence adjust the ultrasound acoustic field accordingly.

In this fashion the system will be able to automatically adjust both acoustic intensity output (to compensate for ultrasound attenuation) and the depth-of-field of the uniform acoustic field (to compensate for narrower or wider skulls) without manual intervention or decision from the first responder personnel.

### Optimal Alignment of Transducers

In another embodiment of the present invention, the transducers 15 may be adhered to the skull using an adhesive (e.g., ultrasound conducting medium 20) that permeates the hair to stick to the scalp for optimal acoustical coupling, without requiring shaving of the hair first. A larger amount of adhesive gives flexibility for the user to press the transducers against the scalp for optimal contact and to push away air pockets. The adhesive may act as a flexible acoustic standoff so that two or more transducers 15 may be aligned (not necessarily in a perpendicular orientation to the skull as skull angles of curvature can vary widely from patient to patient). In one preferred form of the present invention, while a transducer 15 is being affixed to the patient, the signal generator/processor 10 of the present invention is energizing at least one transducer so that that transducer is actively sending out an IS that can be received, i.e., by the opposing transducer in the contralateral attenuation correction embodiment of FIG. 4A. The signal generator processor 10 will monitor and record the amplitude of each IS sent. An audible sound or visual cue on a transducer 15 or signal generator/processor 10 can indicate the relative amplitude of the received signals and hence indicate proper alignment or positioning of the transducers.

Additional means to aid in the proper alignment or positioning of the transducers 15 may include the use of sensors (e.g., accelerometer, imaging, acoustic, etc.) or an external support (e.g., a rigid head frame, flexible shape memory “eyeglasses” frame, etc.). These additional means may be used in conjunction with the transducer alignment scheme discussed above. The sensors may be utilized to ensure proper alignment of, or to locate the optimal bone temporal window for, the placement of the transducers before and during treatment. For example, the use of the IS, or transcranial Doppler, or 2-D transcranial ultrasound, may aid in locating the temporal bone window to allow the transducer to be positioned so as to minimize attenuation of the ultrasound energy. The aforementioned external support (e.g., rigid head frame, flexible shape memory “eyeglasses” frame, etc.) may be used to keep the transducers in place during patient transport.

### Treatment

In a preferred embodiment for ischemic stroke treatment, the desired level of acoustic energy deposited in the region of interest (e.g., the Middle Cerebral Artery) is related to the output power of the transducer 15. However, acoustic output power (i.e., the energy emanating from the transducer before traversing biologic tissue) may or may not be optimal by the time it reaches an ischemic clot, having been affected by factors such as, but not limited to, attenuation or backscattering. In the case of transcranial applications, the desired acoustic energy interacting with a blood clot (and/or with microbubbles present in the region of interest), will be lower than the acoustic energy output at the face of the transducer (e.g., due to attenuation). In order to enable any of the various desired mechanical effects (including, but not limited to, radiation forces, stable cavitation and inertial cavitation) to lyse a clot, the proper amount of acoustic energy must be present within the region of interest, which may be different than the acoustic output energy.

It is well known that bone attenuates ultrasound acoustic intensity and the ability to measure the amount of intensity within a patient's anatomic tissue is currently only available with highly invasive sensor implantation techniques and is impractical and cost-prohibitive (e.g., in the cerebral arteries) in an emergency or pre-hospital setting. It is also well known that acoustic intensity affects microbubbles and, depending on the intensity level, the microbubbles may reflect the ultrasound signal, be in stable or inertial cavitation mode, or impart radiation forces. Also, the type, size and distribution of the microbubbles, or the age, composition and physical characteristics of the clot itself, may be factors affected by the acoustic intensity. Therefore, controlling the amount of acoustic intensity within the region of interest (as opposed to simply the acoustic energy output by the transducer) is desirable and described in the present invention.

Relationship Between Transmitted and Received Signals, Compared to Cadaveric/Preclinical Studies where Peak Negative Pressure was Measured at Midline

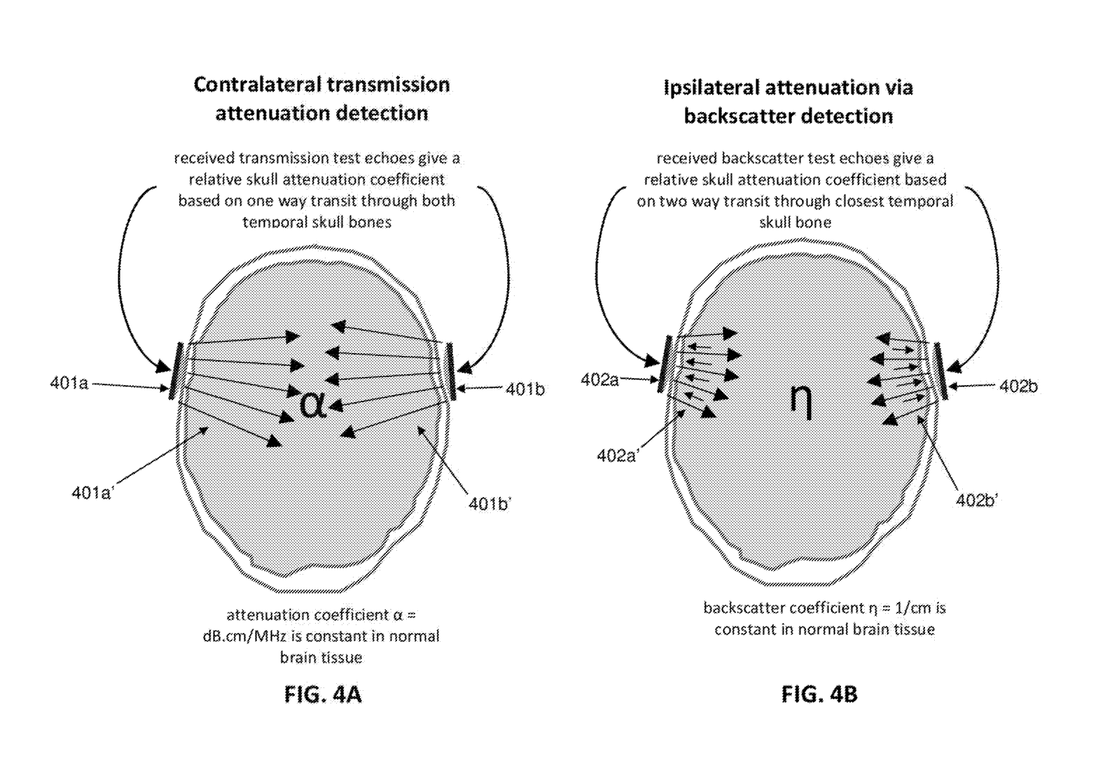

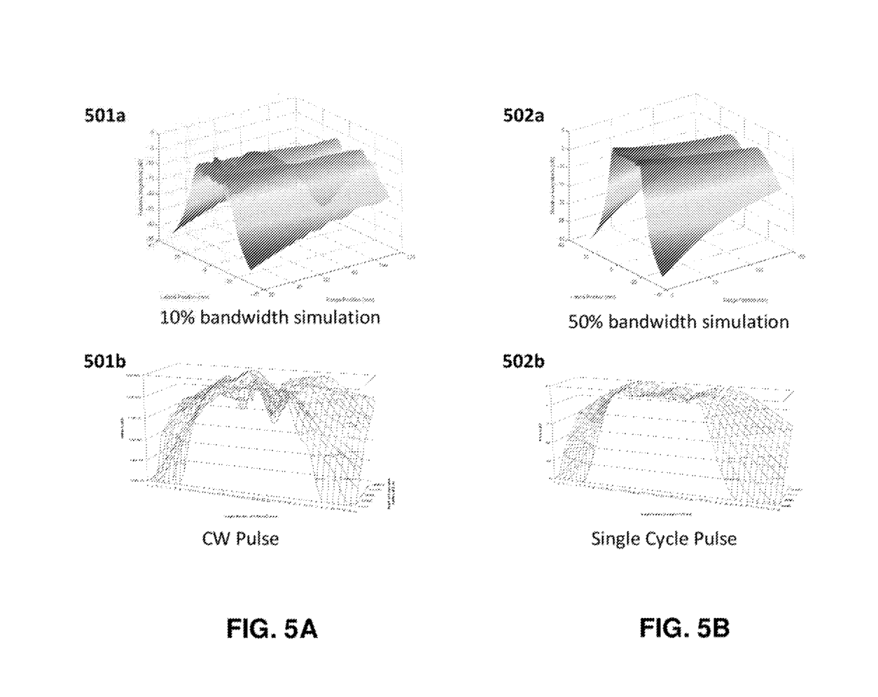

FIG. 5A is a 3-D graphic of a 10% bandwidth simulation 501a and an actual test plot 501b with a narrow bandwidth CW pulse of a beam pattern of ultrasound energy generated by a 25 mm diameter transducer disk of the present invention with a 100 mm convex radius of curvature, operating at 1 MHz (see FIGS. 2A, 2B, 2C and 3). The plots shows the relative magnitude in dB against the depth in mm of the signal. FIG. 5B is a 3-D graphic of a 50% bandwidth simulation 502a and an actual test plot 502b of a broad bandwidth single cycle pulse of a beam pattern of ultrasound energy generated by a 25 mm diameter transducer disk of the present invention with a 100 mm focal length, operating at 1 MHz (see FIGS. 2A, 2B, 2C and 3). By altering factors such as the pulse bandwidth, the present invention is capable of smoothing out the peaks and nulls, especially at the region of interest, which in this example may be between approximately 3-8 cm from the transducer surface and a width of 4 cm diameter at 8 cm depth.

In FIG. 5A, a 10% bandwidth or CW pulse generates a series of energy peaks and nulls represented by the maximum and minimum dB levels throughout the graph, and especially within the region of interest, which in this example, is approximately 3-8 cm (the dark orange areas) from the transducer surface.

FIG. 5B shows a smoother range of energy magnitude (in dBs), especially with the region of interest of approximately 3-8 cm, generated by using a single broadband pulse or changing the pulse bandwidth to 50%. This uniformity in energy deposition is particularly important for clot lysis when treating a stroke patient with a non-imaging, unfocused, diverging ultrasound beam and a cavitation agent (e.g., microbubble) where the location of the clot is not known prior to the treatment. Since neither the use of non-imaging ultrasound nor a pre-treatment head CT scan is needed, this implies that the specific location of the offending clot may be unknown prior to treatment, therefore providing a uniform level of energy (particularly after attenuation or aberration) within the region of interest should permit sufficient energy to reach a clot and achieve the desired effect on the agent. So, for example, if a clot is present in the MCA at a depth of 5 cm from the transducer surface, and microbubbles reach the clot, the ultrasound energy that interacts with the microbubbles may be sufficient to cause stable, inertial cavitation, or radiation force (or a combination of any or all forces) to achieve lysis.

Because multiple pulses of 2-100 msec may be needed for therapy, these would represent narrower bandwidth waveforms more similar to a CW pulse that may exhibit peaks and nulls in the intended ultrasound field. An approach may be employed for more uniform deposition with a “swept and/or stepped frequency” approach where a series of pulses may have increasing frequencies varying around a chosen center frequency. This may prevent peaks and troughs of ultrasound (US) energy forming as would occur in a single frequency long pulse schema. The following method may be used for choosing frequency intervals:
The low frequency should be only half that of the upper frequency in the swept and/or stepped approach. Under these conditions the upper frequency will have twice the nulls as the lower frequency and every other null will match the null of the lower frequency. A design can then use as many steps in between, as more steps will smooth the average beamplot to a greater degree. A subtle enhancement to this is that it is best not to use the upper frequency in the sequence since this will reinforce the nulls in the lower frequency. These steps to calculate the enhanced method frequencies now proceed as follows:

Let:

FL=Low Frequency

FU=Unused High Frequency

F=Center Frequency=1 MHz

A=Step Size

N=Step Multiplier

FH=High Frequency=FL+(2N−1)*A

Then

F−(N−0.5)A=FL

F+(N+0.5)A=FU

FU=2FL

F+(N+0.5)A=2FL

F+(N+0.5)A=2(F−(N−0.5)A)

F+(N+0.5)A=2F−2(N−0.5)A

(N+0.5)A+2(N−0.5)A=F

[(N+0.5)+2(N−0.5)]A=F

[N+0.5+2N−1]A=F

[3N−0.5]A=F

A=F/(3N−0.5)
As an example, At 1 MHz and N=2:
A=0.18182 MHz
f=[0.72727 0.90909 1.0909 1.2727] MHz
FU=1.4545
   Free field (i.e., no attenuation media) water tank experiments demonstrate the practical effects of this frequency smoothing technique.

Microbubble States

Some researchers have postulated that stable cavitation is the primary means by which ultrasound alone, or with injected microbubbles, can assist in the lysing of thrombus. However, simulations of actual clinical trial studies using ultrasound and tPA suggest that successful ultrasound-mediated clot lysis did not have adequate transmitted energy through the skull to achieve a mechanical index sufficient to reach any stable cavitation levels (Ultrasound Med Biol. 2009 July; 35(7):1148-58). In vitro work points to other potential mechanisms where ultrasound alone, or the interaction of ultrasound with injected microbubbles, can achieve a lysing effect. The mechanisms by which ultrasound can assist in the lysing of thrombotic occlusions is multivariate and can include, simultaneously, any or all of the below mechanical processes:
Ultrasound interaction with the thrombus that increases endogenous tPA effectiveness at breaking down fibrin;
Stable cavitation of injected microbubbles that provides microstreaming energy at the periphery of the thrombus;
Inertial cavitation of injected microbubbles that provides microjets at the periphery of or internal to the thrombus; and/or
Radiation energy of the ultrasound beam which forces the injected microbubbles to tunnel through the fibrin/platelet matrix in the thrombus, exposing more surface area and weakening the bonds holding the thrombus together.

In all of these processes, the injected microbubbles are not taken up metabolically in any chemical interaction during clot lysis via ultrasound. In addition, ultrasound excitation of endothelial tissue can release nitric oxide to produce vasodilation and provide beneficial collateral blood flow to affected brain tissue.

The ability of an ultrasound beam of a given acoustic intensity or mechanical index to achieve either stable or inertial cavitation, or to be impacted by radiation forces, is directly related to the diameter of the microbubble and its elasticity. Because commercially-available microbubbles are heterogeneous in size or “polydisperse”, an ultrasound beam with a given acoustic intensity or mechanical index in a volume of the brain may simultaneously induce cavitation, stable cavitation, inertial cavitation and/or radiation forces to a subset of the circulating microbubbles in that region of interest. These would provide a cumulative effect on dissolving thrombus.

Reduced Risk of Standing Waves

In an intracranial application of the present invention, the divergence of the ultrasound beam may still diminish the acoustic energy that reaches the contralateral skull wall but will be somewhat more concentrated than for a widely divergent beam. However, when one considers the attenuation of ultrasound in brain tissue that is frequency dependent, a 1 MHz transducer beam will be attenuated at a 5× faster rate than, for example, a 200 kHz transducer reducing the potential for unintended consequences created by far side reflections or standing waves (Table 1).

TABLE 1
Attenuation Comparison of a 1 MHZ vs. 200 kHz acoustic
beam (assuming similar beam geometry and an equivalent
20 dB signal starting at 0 cm deep). Brain tissue
attenuation ≈ −0.8 dB/cm/MHz.

| Depth | 1.0 MHz | 200 kHz |
|---|---|---|
| 3 cm | 17.6 dB | 19.5 dB |
| 5 cm | 16.0 dB | 19.2 dB |
| 16 cm | 7.2 dB | 17.4 dB |

Some existing approaches rely on natural beam-spreading from transducers operating at very low frequencies, e.g., at 200 kHz, to achieve a wide field of view (FOV). In an embodiment of the present invention, a convex PZT transducer 15 (e.g., such as is shown in FIGS. 2A, 2B and 2C) operating at approximately 1 MHz provides optimal FOV for covering cerebral vessels most likely to cause stroke while not creating standing waves. The more narrowly diverging beam provided by the present invention will provide more uniform acoustic energy in the region of interest but will still fall off in intensity with the square of the distance as it approaches the far skull wall, thus reducing the risk of rebound interference. Also, a 1.0 MHz frequency will experience greater attenuation as it travels through brain tissue. Thus, a 1.0 MHz beam at 8 cm deep will continue to attenuate more rapidly in brain tissue before it strikes the far skull wall than an equivalent energy 200 kHz beam at 8 cm deep.

Also, with a potentially highly reflective opposite skull, there is a risk of interference, constructive and destructive whenever the pulse is reflected and propagates back across the initial forward therapeutic wave. This can occur at any time later than the time taken to propagate across the skull. As the sound velocity in brain tissue is approximately 1540 m/sec, a 100 msec pulse will have a pulse length of 154 meters. A 2 msec pulse will have a pulse length of 3.08 m. One needs to be concerned with standing waves that can occur in an enclosed space, such as the skull. Standing waves take several passes to across the skull to develop and can reach much greater magnitudes than exist in the original forward wave. Attenuation of the tissue makes this risk negligible. The attenuation in brain tissue is on the order of 0.8 dB/cm/MHz. At 3 m and 1 MHz, the leading edge of a 2 msec pulse will be attenuated by 240 dB and will contribute negligible interference. Due to this beneficial attenuation, the interference issues with a 100 msec pulse are not significantly greater than the interference with a 2 msec pulse.

Acoustic power output and the resultant energy deposition within the region of interest, however, can only be of use if the specific attenuation characteristics of the intervening skull can be estimated and corrected for in real-time. If the depth at which the clot is lodged is found, e.g., by other diagnostic tests, an intentionally higher peak energy can be provided with the single diverging transducer to more aggressively treat that now-located occluding clot.

The current standard of care (if previous efforts to dissolve the clot have not been successful) is to use increasingly aggressive procedures, such as intra-arterial administration of tPA and catheter-based thrombectomy devices, to remove a clot. The present invention provides similar ability to increase, or “dial up”, the intensity of treatment, especially if a clot is not lysed using initial settings. The multiple pulse characteristics can be selected which can provide an intentional peak energy node at a specified depth. By this method an even higher peak negative pressure can be deposited within a relatively narrow band at a specified depth corresponding to the location of the clots as found on X-ray or magnetic resonance (MR) angiography.

### Microbubbles

In one preferred form of the invention, microbubbles 35 are used in conjunction with ultrasound thrombolysis system 5. Microbubbles 35 may comprise substantially any biocompatible microbubble composition which can facilitate lysing of a blood clot when excited by ultrasound energy. In one preferred form of the invention, microbubbles 35 comprise a gaseous component, e.g., perflexane, and a phospholipid membrane component, such as 1,2-dimyristoyl-sn-glycero-3-phosphocholine (DMPC) with a size distribution of 79% <3 μm and 21% 3-10 μm, or human serum albumin and perflutren with a mean diameter or 3.0-4.5 μm.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. An ultrasound system for providing therapy to a site in a patient, the ultrasound system comprising:
at least one transducer configured for removable securement to a patient; and
a signal generator/processor configured for removable connection to the at least one transducer, the signal generator/processor being configured to apply an electrical signal to the at least one transducer so as to cause the at least one transducer to deliver ultrasound energy to the site in the patient, whereby to provide therapy to the site in the patient;
wherein the signal generator/processor is configured to (i) identify ultrasound attenuation as the ultrasound energy passes through the patient, and (ii) modify the electrical signal applied to the at least one transducer so as to compensate for ultrasound attenuation in the patient, whereby to deliver a pre-determined level of ultrasound energy to the site in the patient.

2. An ultrasound system according to claim 1 wherein the signal generator/processor is configured to (i) automatically identify ultrasound attenuation in the patient, and (ii) automatically modify the electrical signal applied to the at least one transducer so as to compensate for ultrasound attenuation, whereby to automatically deliver a pre-determined level of ultrasound energy to the site in the patient.

3. An ultrasound system according to claim 1 wherein the signal generator/processor is configured to (a) apply an electrical interrogation signal to the at least one transducer so as to cause the at least one transducer to deliver an ultrasound interrogation signal to the patient, (b) detect the ultrasound interrogation signal after the ultrasound interrogation signal has passed through at least a portion of the patient, (c) determine the level of ultrasound attenuation from the detected ultrasound interrogation signal, and (d) compensate for ultrasound attenuation by adjusting the electrical signal applied to the at least one transducer so as to deliver a pre-determined level of ultrasound energy to the site in the patient.

4. An ultrasound system according to claim 3 wherein the pre-determined level of ultrasound energy is sufficient to trigger at least one mechanism of action at the site.

5. An ultrasound system according to claim 4 wherein the at least one mechanism of action is at least one from the group consisting of: stable cavitation, inertial cavitation and radiation force.

6. An ultrasound system according to claim 3 wherein the signal generator/processor compares the detected ultrasound interrogation signal with an attenuation table so as to determine the level of ultrasound attenuation in the patient.

7. An ultrasound system according to claim 6 wherein the attenuation table is generated by at least one from the group consisting of cadaveric studies and calibration block studies.

8. An ultrasound system according to claim 1 wherein the signal generator/processor compensates for ultrasound attenuation by adjusting at least one of the following parameters of the electrical signal applied to the at least one transducer: frequency, bandwidth, voltage, output energy, intensity, pulse length and duty cycle.

9. An ultrasound system according to claim 3 wherein the ultrasound system comprises an ipsilateral transducer and a contralateral transducer, and further wherein the signal generator/processor is configured to apply the electrical interrogation signal to the ipsilateral transducer and to detect the ultrasound interrogation signal with the contralateral transducer.

10. An ultrasound system according to claim 9 wherein the signal generator/processor is configured to provide an electrical interrogation signal to the ipsilateral transducer, and further wherein the signal generator/processor is configured to generate an audible signal or visual display corresponding to the detected ultrasound interrogation signal for use as a cue in aligning the ipsilateral and contralateral transducers.

11. An ultrasound system according to claim 9 wherein the signal generator/processor is also configured to apply an electrical interrogation signal to the contralateral transducer and to detect an ultrasound interrogation signal with the ipsilateral transducer, and further wherein the signal generator/processor is configured to average the detected ultrasound interrogation signals.

12. An ultrasound system according to claim 9 wherein the signal generator/processor is configured to determine (i) the size of the patient's anatomy between the ipsilateral transducer and the contralateral transducer, and (ii) modify the electrical signal applied to the at least one transducer based on the size of the patient's anatomy.

13. An ultrasound system according to claim 9 wherein the signal generator/processor is configured to determine the size of the patient's anatomy between the ipsilateral transducer and the contralateral transducer by measuring the time-of-flight of the ultrasound interrogation signal from the ipsilateral transducer to the contralateral transducer.

14. An ultrasound system according to claim 3 wherein the ultrasound system comprises an ipsilateral transducer, and further wherein the signal generator/processor is configured to apply the electrical interrogation signal to the ipsilateral transducer so as to generate the ultrasound interrogation signal and to detect the returning/reflected ultrasound interrogation signal with the same ipsilateral transducer after the ultrasound interrogation signal has traversed some portion of the patient's body.

15. An ultrasound system according to claim 1 further comprising a frame for removably securing the at least one transducer to a patient.

16. An ultrasound system according to claim 1 further comprising an adhesive for removably securing the at least one transducer to a patient.

17. An ultrasound system according to claim 16 wherein the adhesive comprises a low attenuation ultrasound-conducting medium.

18. An ultrasound system according to claim 17 wherein the adhesive comprises at least one of the following: a hydrogel, olyoxyethylene 20 cetyl ether, water, glycerin, calcium carbonate, 1,2 propanediol, polyoxyethylene 20 sorbitol, and methylparaben or propylparaben, woven fabric, plastic (PVC, polyethylene or polyurethane) or latex.

19. An ultrasound system according to claim 1 wherein the site comprises a blood clot, and further wherein the ultrasound system delivers ultrasound energy to the blood clot so as to lyse the blood clot.

20. An ultrasound system according to claim 19 wherein the blood clot is located in the brain.

21. An ultrasound system according to claim 19 wherein the blood clot is located in a coronary artery.

22. An ultrasound system according to claim 19 wherein the blood clot is located in a peripheral artery.

23. An ultrasound system according to claim 1 wherein the site comprises an interface between a circulatory pathway and tissue, and further wherein the system delivers ultrasound energy to the interface so as to enhance drug delivery to the tissue.

24. An ultrasound system according to claim 23 wherein the interface comprises the blood brain barrier.

25. An ultrasound system according to claim 1 wherein the at least one transducer is configured for removable securement to the head of the patient.

26. An ultrasound system according to claim 1 wherein the at least one transducer is configured for removable securement to the chest of the patient.

27. An ultrasound system according to claim 1 wherein the at least one transducer is configured for removable securement to a limb of the patient.

28. An ultrasound system according to claim 1 wherein the signal generator/processor is configured for removable connection to the at least one transducer via a cable.

29. An ultrasound system according to claim 1 wherein the signal generator/processor is configured for removable connection to the at least one transducer via a wireless connection.

30. An ultrasound system according to claim 1 wherein the signal generator/processor is configured so that it may be disconnected from the at least one transducer after the at least one transducer has been removably secured to the patient, and further wherein the disconnected signal generator/processor may thereafter be replaced by a replacement signal generator/processor.

31. An ultrasound system according to claim 1 wherein the at least one transducer and the signal generator/processor are configured to provide the ultrasound energy in the form of a substantially uniform diverging beam so as to enhance the therapy delivered to the site in the patient.

32. An ultrasound system according to claim 31 wherein the at least one transducer comprises a component having a convex configuration.

33. An ultrasound system according to claim 32 wherein the component comprises a piezoelectric material.

34. An ultrasound system according to claim 33 wherein the piezoelectric material is diced into posts and then filled with epoxy so as to form a convex configuration.

35. An ultrasound system according to claim 33 further comprising a second component disposed adjacent to the first component, wherein the second component comprises an inverted concave acoustic lens.

36. An ultrasound system according to claim 31 wherein the at least one transducer has a bandwidth of approximately 30% or greater.

37. An ultrasound system according to claim 31 wherein the signal generator/processor is configured to adjust the electrical signal applied to the at least one transducer in accordance with patient-specific variables so as to provide a substantially uniform diverging beam.

38. An ultrasound system according to claim 37 wherein the depth range of the substantially uniform diverging beam is adjusted by modifying the frequency bandwidth and pulse characteristics of the electrical signal applied to the at least one transducer.

39. An ultrasound system according to claim 31 wherein the depth range of the substantially uniform diverging beam is approximately 3-8 cm deep.

40. An ultrasound system according to claim 39 wherein the substantially uniform diverging beam has a spread of approximately 4 cm at a depth of approximately 8 cm deep.

41. An ultrasound system according to claim 31 wherein the signal generator/processor is configured to automatically adjust at least one of the following parameters of the electrical signal so as to provide a substantially uniform diverging beam: frequency, phase, timing and pulse parameters, including output energy, bandwidth, voltage, intensity, pulse length, and duty cycle.

42. An ultrasound system according to claim 41 wherein the electrical signal has a swept and/or stepped frequency around a chosen center frequency so as to minimize peaks and/or nulls in the substantially uniform diverging beam.

43. An ultrasound system according to claim 31 wherein the signal generator/processor is configured to apply a succession of single half-cycle or full-cycle pulses to the at least one transducer.

44. An ultrasound system according to claim 1 wherein the at least one transducer and the signal generator/processor are configured to provide the ultrasound energy in the form of a focused beam.

45. An ultrasound system according to claim 1 wherein the at least one transducer and the signal generator/processor are provided in the form of a prepackaged kit.

46. An ultrasound system according to claim 1 further comprising an agent for delivery to the site in the patient.

47. An ultrasound system according to claim 46 wherein the agent enhances lysing of a blood clot when the agent is disposed in the vicinity of the blood clot and exposed to ultrasound energy.

48. An ultrasound system according to claim 46 wherein the agent enhances the permeability of an interface between a circulatory pathway and tissue.

49. An ultrasound system according to claim 48 wherein the interface comprises the blood brain barrier.

50. An ultrasound system according to claim 47 wherein the agent comprises microbubbles, nanoparticles or drugs.

51. An ultrasound system according to claim 46 wherein the at least one transducer, the signal generator/processor and the agent are provided in the form of a prepackaged kit.

52. An ultrasound system according to claim 1 wherein the signal generator/processor is configured to collect event data.

53. An ultrasound system according to claim 52 wherein the at least one transducer comprises a unique identifier, and further wherein the event data collected by the signal generator/processor is tagged with the unique identifier.

54. An ultrasound system according to claim 52 wherein the event data comprises at least one from the group consisting of date or time, global positioning system (GPS), accelerometer, sensor, biological, chemical, demographic, patient-specific, functional or cognitive test information, or other data.

55. An ultrasound system according to claim 52 wherein the signal generator/processor is configured to transmit the collected event data.

56. An ultrasound system according to claim 55 wherein the collected event data is transmitted to a medical registry.

57. An ultrasound system according to claim 56 wherein the medical registry contains aggregated data from a plurality of patients.

58. An ultrasound system according to claim 1 wherein the signal generator/processor is configured to receive instructions from a remote location in order to change its operating parameters.

59. An ultrasound system for providing therapy to a site in a patient, the ultrasound system comprising:
at least one transducer configured for removable securement to a patient; and
a signal generator/processor configured for removable connection to the at least one transducer, the signal generator/processor being configured to apply an electrical signal to the at least one transducer so as to cause the at least one transducer to deliver ultrasound energy to the site in the patient, whereby to provide therapy to the site in the patient;
wherein the at least one transducer and the signal generator/processor are configured to provide the ultrasound energy to the site in the patient in the form of a substantially uniform diverging beam.

60. A system for delivering ultrasound to a patient, the system comprising:
at least one transducer configured for removable securement to a patient; and
an adhesive for removably securing the at least one transducer to a patient;
wherein the at least one transducer and the adhesive are provided as a prepackaged kit.

61. A system according to claim 60 wherein the at least one transducer and the adhesive are provided as an integrated device.

62. A system according to claim 60 wherein the adhesive contains or comprises a low attenuation ultrasound-conducting medium.

63. A system according to claim 62 wherein the adhesive comprises at least one of the following: a hydrogel, olyoxyethylene 20 cetyl ether, water, glycerin, calcium carbonate, 1,2 propanediol, polyoxyethylene 20 sorbitol, and methylparaben or propylparaben.

64. A system according to claim 60 wherein the system further comprises a signal generator/processor configured for removable connection to the at least one transducer, the signal generator/processor being configured to apply an electrical signal to the at least one transducer so as to cause the at least one transducer to deliver ultrasound energy to the site in the patient, whereby to provide therapy to the site in the patient.

65. A method for providing therapy to a site in a patient, the method comprising:
providing an ultrasound system for providing therapy to a site in a patient, the ultrasound system comprising:
at least one transducer configured for removable securement to a patient; and
a signal generator/processor configured for removable connection to the at least one transducer, the signal generator/processor being configured to apply an electrical signal to the at least one transducer so as to cause the at least one transducer to deliver ultrasound energy to the site in the patient, whereby to provide therapy to the site in the patient;
wherein the signal generator/processor is configured to (i) identify ultrasound attenuation as the ultrasound energy passes through the patient, and (ii) modify the electrical signal applied to the at least one transducer so as to compensate for ultrasound attenuation in the patient, whereby to deliver a pre-determined level of ultrasound energy to the site in the patient;
removably securing the at least one transducer to a patient, and removably connecting the signal generator/processor configured to the at least one transducer; and
applying an electrical signal to the at least one transducer with the signal generator/processor so as to cause the at least one transducer to deliver ultrasound energy to the site in the patient, whereby to provide therapy to the site in the patient.

66. A method for providing therapy to a site in a patient, the method comprising:
providing an ultrasound system for providing therapy to a site in a patient, the ultrasound system comprising:
at least one transducer configured for removable securement to a patient; and
a signal generator/processor configured for removable connection to the at least one transducer, the signal generator/processor being configured to apply an electrical signal to the at least one transducer so as to cause the at least one transducer to deliver ultrasound energy to the site in the patient, whereby to provide therapy to the site in the patient;
wherein the at least one transducer and the at least one signal generator/processor are configured to provide the ultrasound energy to the site in the patient in the form of a substantially uniform diverging beam;
removably securing the at least one transducer to a patient, and removably connecting the signal generator/processor configured to the at least one transducer; and
applying an electrical signal to the at least one transducer with the signal generator/processor so as to cause the at least one transducer to deliver ultrasound energy to the site in the patient, whereby to provide therapy to the site in the patient.

67. A method for providing therapy to a site in a patient, the method comprising:
providing an ultrasound system for providing therapy to a site in a patient, the ultrasound system comprising:
at least one transducer configured for removable securement to a patient; and
an adhesive for removably securing the at least one transducer to a patient;
wherein the at least one transducer and the adhesive are provided as a prepackaged kit;
opening the prepackaged kit;
applying the adhesive and the at least one transducer to a patient; and
using the at least one transducer to deliver ultrasound energy to the site in the patient, whereby to provide therapy to the site in the patient.

68. A method according to claim 67 wherein the adhesive is applied to the patient and then the at least one transducer is applied to the adhesive.

69. A method according to claim 67 wherein the adhesive is applied to the at least one transducer, and the adhesive and the at least one transducer are applied to the patient.

70. A method according to claim 69 wherein the adhesive is applied to the at least one transducer before the prepackaged kit is sealed.

71. A method according to claim 69 wherein the adhesive is applied to the at least one transducer after the prepackaged kit is opened.
